(19) United States
(12) Patent Application Publication
(10) Pub. No.: US 2009/0036465 A1
(43) Pub. Date: Feb. 5, 2009

(54) COMBINATION THERAPY FOR PULMONARY ARTERIAL HYPERTENSION

(75) Inventors: Robert ROSCIGNO, Chapel Hill, NC (US); Eugene SULLIVAN, Olney, MD (US)

Correspondence Address: FOLEY AND LARDNER LLP;SUITE 500, 3000 K STREET NW, WASHINGTON, DC, 20007, US

(73) Assignee: United Therapeutics Corporation

(21) Appl. No.: 11/873,645

(22) Filed: Oct. 17, 2007

Related U.S. Application Data
(60) Provisional application No. 60/852,395, filed on Oct. 18, 2006.

(51) Int. Cl. A61K 31/192; A61P 9/12; A61K 31/44

(52) U.S. Cl. 514/253.05; 514/569; 514/277

(57) ABSTRACT

Methods for treatment of pulmonary hypertension include administration of a first amount of treprostinil and administration of a second amount of a Rho kinase inhibitor such that the first amount and the second amount form together an amount effective for pulmonary hypertension treatment. Kits for treatment pulmonary hypertension are also disclosed.

### RELATED APPLICATIONS

The present application claims priority to U.S. provisional application No. 60/852,395 filed Oct. 18, 2006, which is incorporated herein by reference in its entirety.

### FIELD

The present application relates to treatment of pulmonary hypertension and, in particular, to treatment of pulmonary hypertension using a combination of Treprostinil and a Rho kinase inhibitor.

### BACKGROUND

All blood is driven through the lungs via the pulmonary circulation in order, among other things, to replenish the oxygen which it dispenses in its passage around the rest of the body via the systemic circulation. The flow through both circulations is in normal circumstances equal, but the resistance offered to it in the pulmonary circulation is generally much less than that of the systemic circulation. When the resistance to pulmonary blood flow increases, the pressure in the circulation is greater for any particular flow. This is referred to as pulmonary hypertension (PH). Generally, pulmonary hypertension is defined through observations of pressures above the normal range pertaining in the majority of people residing at the same altitude and engaged in similar activities.

Most often pulmonary hypertension is a manifestation of an obvious or explicable increase in resistance, such as obstruction to blood flow by pulmonary emboli, malfunction of the heart's valves or muscle in handling blood after its passage through the lungs, diminution in pulmonary vessel caliber as a reflex response to hypoventilation and low oxygenation, or a mismatch of vascular capacity and essential blood flow, such as shunting of blood in congenital abnormalities or surgical removal of lung tissue. Such pulmonary hypertension is referred to as secondary hypertension.

There remain some cases of pulmonary hypertension where the cause of the increased resistance is as yet inexplicable. They are described as idiopathic (primary) pulmonary hypertension (PPH) and are diagnosed by and after exclusion of the causes of secondary pulmonary hypertension. Despite the possibility of a varied etiology, cases of idiopathic pulmonary hypertension tend to comprise a recognizable entity. Approximately 65% of the most commonly afflicted are female and young adults, though it has occurred in children and patients over 50. Life expectancy from the time of diagnosis is short, about 3 to 5 years, though occasional reports of spontaneous remission and longer survival are to be expected given the nature of the diagnostic process. Generally, however, progress is inexorable via syncope and right heart failure and death is quite often sudden.

Pulmonary hypertension refers to a condition associated with an elevation of pulmonary arterial pressure (PAP) over normal levels. In humans, a typical mean PAP is approximately 12-15 mm Hg. Pulmonary hypertension, on the other hand, is sometimes marked by PAP increases by at least 5 to 10 mm Hg over normal levels. PAP readings as high as 50 to 100 mm Hg over normal levels have been reported. When the PAP markedly increases, plasma can escape from the capillaries into the lung interstitium and alveoli. Fluid buildup in the lung (pulmonary edema) can result, with an associated decrease in lung function that can in some cases be fatal.

Pulmonary hypertension may either be acute or chronic. Acute pulmonary hypertension is often a potentially reversible phenomenon generally attributable to constriction of the smooth muscle of the pulmonary blood vessels, which may be triggered by such conditions as hypoxia (as in high-altitude sickness), acidosis, inflammation, or pulmonary embolism. Chronic pulmonary hypertension is characterized by major structural changes in the pulmonary vasculature, which result in a decreased cross-sectional area of the pulmonary blood vessels. This may be caused by, for example, chronic hypoxia, thromboembolism, or unknown causes (idiopathic or primary pulmonary hypertension).

Pulmonary hypertension has been implicated in several life-threatening clinical conditions, such as adult respiratory distress syndrome (“ARDS”) and persistent pulmonary hypertension of the newborn (“PPHN”). Zapol et. al., Acute Respiratory Failure, p. 241-273, Marcel Dekker, New York (1985); Peckham, J. Ped. 93:1005 (1978). PPHN, a disorder that primarily affects full-term infants, is characterized by elevated pulmonary vascular resistance, pulmonary arterial hypertension, and right-to-left shunting of blood through the patent ductus arteriosus and foramen ovale of the newborn's heart. Mortality rates range from 12-50%. Fox, Pediatrics 59:205 (1977); Dworetz, Pediatrics 84:1 (1989). Pulmonary hypertension may also result in a potentially fatal heart condition known as “cor pulmonale,” or pulmonary heart disease. Fishman, “Pulmonary Diseases and Disorders” 2^{nd} Ed., McGraw-Hill, New York (1988).

FDA approved treatments for pulmonary hypertension include three prostanoids (epoprostenol, iloprost and treprostinil), one endothelin receptor antagonist (bosentan) and one phosphodiesterase-5 inhibitor (sildenafil). In addition, although not approved by the FDA for this use, diuretics, calcium channel blockers, anticoagulants, and digoxin have also been utilized for PAH for a number of years, see e.g. Badesch D B, Abman S H, Ahearn G S, et al. 2004. Medical therapy for pulmonary arterial hypertension: ACCP evidence-based clinical practice guidelines. Chest. 126:35-62. Despite a certain success achieved in recent years, a response to FDA approved therapies of PAH is often incomplete, and many patients will experience progression of disease on therapy, see e.g. Rubin L J and Galie N. 2004. Pulmonary arterial hypertension: a look to the future. J Am Coll Cardiol. 43:89-90. Thus, a need exists to develop alternative therapies for treatment of pulmonary hypertension.

### SUMMARY

According to one embodiment, a method of treating pulmonary hypertension comprises administering to a subject in need thereof (A) a first amount of Treprostinil or its derivative, or a pharmaceutically acceptable salt thereof, and (B) a second amount of a Rho Kinase inhibitor or its derivative or a pharmaceutically acceptable salt thereof, wherein the first amount and the second amount together comprise an amount effective for treatment of pulmonary hypertension.

According to another embodiment, a kit for treating pulmonary hypertension comprises (i) a first amount of Treprostinil, or its derivative, or a pharmaceutically acceptable salt thereof, (ii) a second amount of a Rho kinase inhibitor or its derivative or a pharmaceutically acceptable salt thereof, and (iii) instructions for use in treating pulmonary hypertension, wherein the first amount and the second amount comprise together an amount effective for treatment of pulmonary hypertension.

### DETAILED DESCRIPTION

Unless otherwise specified, the term “a” or “an” used herein shall mean “one or more.”

The inventors believe that treprostinil does not affect Rho kinase activity. Thus, a combination of treprostinil and Rho Kinase inhibitor can provide a more complete and/or more universal treatment of pulmonary hypertension than treprostinil treatment or Rho Kinase inhibitor treatment alone because treprostinil and a Rho Kinase inhibitor can act by distinct, complementary mechanisms. The administration of combination of treprostinil and a Rho Kinase inhibitor may lower doses of treprostinil and the Rho Kinase inhibitor compared to administering treprostinil and Rho Kinase Inhibitor alone and thus may allow avoiding dose-related adverse side effects. For example, Tawara et al. provides evidence that a combined therapy of a Rho-Kinase inhibitor and an oral prostacyclin analogue, beraprost sodium (BPS), may be more effective for ameliorating monocrataline-induced pulmonary hypertension in rats than either of the Rho-Kinase inhibitor or BPS alone, see Cardiovasc. Pharmacol., vol. 50(2), p. 195-200, 2007.

Accordingly, one embodiment provides a method of treating pulmonary hypertension comprising administering to a subject in need thereof a first amount of Treprostinil or its derivative or a pharmaceutically acceptable salt thereof and administering a second amount of a Rho Kinase inhibitor or its derivative or a pharmaceutically acceptable salt thereof, such that the first amount and the second amount comprise together an amount effective for treatment of pulmonary hypertension. The administration of the first amount and the second amount can be performed separately, sequentially or simultaneously.

Suitable derivatives of Treprostinil include acid derivatives, pro-drugs, sustained release forms, inhaled forms and oral forms of Treprostinil, including those disclosed in U.S. Pat. No. 6,521,212 and US patent publications Nos. 20050085540 and 20050282901. Suitable derivatives of Rho Kinase inhibitor include derivatives, pro-drugs, sustained release forms, inhaled forms, oral forms and forms for parenteral administration such as intravenous or subcutaneous administration.

Treprostinil, or 9-deoxy-2′,9-alpha-methano-3-oxa-4,5,6-trinor-3,7-(1′3′-interphenylene)-13,14-dihydro-prostaglandin F₁, is a prostacyclin analogue, first described in U.S. Pat. No. 4,306,075. U.S. Pat. No. 5,153,222 describes use of treprostinil for treatment of pulmonary hypertension. Treprostinil is approved for the intravenous as well as subcutaneous route, the latter avoiding septic events associated with continuous intravenous catheters. U.S. Pat. Nos. 6,521,212 and 6,576,033 describe administration of treprostinil by inhalation for treatment of pulmonary hypertension. U.S. provisional application No. 60/800,016 filed May 15, 2006, describes administration of treprostinil by inhalation using metered dose inhalers for treatment of pulmonary hypertension.

The term “acid derivative” is used herein to describe C1-4 alkyl esters and amides, including amides wherein the nitrogen is optionally substituted by one or two C1-4 alkyl groups.

The present invention also encompasses methods of using Treprostinil or its derivatives, or pharmaceutically acceptable salts thereof. In one embodiment, a method uses Treprostinil sodium, currently marketed under the trade name of REMODULIN®. The FDA has approved Treprostinil sodium for the treatment of pulmonary arterial hypertension by injection of dose concentrations of 1.0 mg/mL, 2.5 mg/mL, 5.0 mg/mL and 10.0 mg/mL. The chemical structure formula for Treprostinil sodium is:

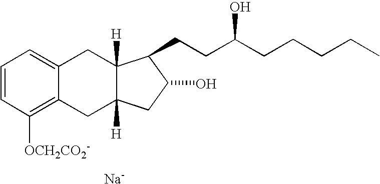

Treprostinil sodium is sometimes designated by the chemical names: (a) [(1R,2R,3aS,9aS)-2,3,3a,4,9,9a-hexahydro-2-hydroxy-1-[(3S)-3-hydroxyoctyl]-1H-benz[ƒ]inden-5-yl]oxy]acetic acid; or (b) 9-deoxy-2′,9-α-methano-3-oxa-4,5,6-trinor-3,7-(1′,3′-interphenylene)-13,14-dihydro-prostaglandin F₁. Treprostinil sodium is also known as: UT-15; LRX-15; 15AU81; UNIPROST™; BW A15AU; and U-62,840. The molecular weight of Treprostinil sodium is 390.52, and its empirical formula is C₂₃H₃₄O₅.

The present invention extends to methods of using physiologically acceptable salts of Treprostinil, as well as non-physiologically acceptable salts of Treprostinil that may be used in the preparation of the pharmacologically active compounds of the invention.

Physiologically acceptable salts of Treprostinil include salts derived from bases. Base salts include ammonium salts (such as quaternary ammonium salts), alkali metal salts such as those of sodium and potassium, alkaline earth metal salts such as those of calcium and magnesium, salts with organic bases such as dicyclohexylamine and N-methyl-D-glucamine, and salts with amino acids such as arginine and lysine.

Quaternary ammonium salts can be formed, for example, by reaction with lower alkyl halides, such as methyl, ethyl, propyl, and butyl chlorides, bromides, and iodides, with dialkyl sulphates, with long chain halides, such as decyl, lauryl, myristyl, and stearyl chlorides, bromides, and iodides, and with aralkyl halides, such as benzyl and phenethyl bromides.

The term “acid derivative” is used herein to describe C1-4 alkyl esters and amides, including amides wherein the nitrogen is optionally substituted by one or two C1-4 alkyl groups.

The invention also includes bioprecursors or “pro-drugs” of Treprostinil, that is, compounds which are converted in vivo to Treprostinil or its pharmaceutically active derivatives thereof.

The amount of Treprostinil or its derivative, or a pharmaceutically acceptable salt thereof, that is required in a medication or diagnostic aid according to the invention to achieve the desired effect will depend on a number of factors, such as the specific application, the nature of the particular compound used, the mode of administration, the concentration of the compound used, and the weight and condition of the patient. A daily dose per patient may be in the range 25 μg to 250 mg; 0.5 μg to 2.5 mg, or 7 μg to 285 μg. per day per kilogram bodyweight. For example, an intravenous dose in the range 0.5 μg to 1.5 mg per kilogram bodyweight per day may conveniently be administered as an infusion of from 0.5 ng to 1.0 μg per kilogram bodyweight per minute. One possible dosage is 2.5 ng/kg/min, increased over 12 weeks by an amount of 2.50 ng/kg/min each week, until a target dose, such as 15 ng/kg/min, is reached. Infusion fluids suitable for this purpose contain, for example, from 10 ng to 1 μg per milliliter. Ampoules for injection contain, for example, from 0.1 μg to 1.0 mg and orally administrable unit dose formulations, such as tablets or capsules, contain, for example, from 0.1 to 100 mg, typically from 1 to 50 mg. For diagnostic purposes, a single unit dose formulation may be administered. In the case of physiologically acceptable salts, the weights indicated above refer to the weight of the active compound ion, that is, the ion derived from Treprostinil.

In the manufacture of a medicament or diagnostic aid according to the invention, hereinafter referred to as a “formulation,” Treprostinil and/or its derivatives, and/or pharmaceutically acceptable salts thereof, may be admixed with, inter alia, an acceptable carrier. The carrier must, of course, be acceptable in the sense of being compatible with any other ingredients in the formulation and must not be deleterious to the subject. The carrier may be a solid or a liquid, or both, and is preferably formulated with the compound as a unit-dose formulation, for example, a tablet, which may contain from 0.05% to 95% by weight of the active compound. One or more of Treprostinil or its derivatives, or pharmaceutically acceptable salts thereof, may be incorporated in the formulations of the invention, which may be prepared by any of the well known techniques of pharmacy for admixing the components.

The formulations of the invention include those suitable for parenteral (e.g., subcutaneous, intramuscular, intradermal, or intravenous), oral, inhalation (in solid and liquid forms), rectal, topical, buccal (e.g., sub-lingual) and transdermal administration.

Formulations of the present invention suitable for parenteral administration conveniently comprise sterile aqueous preparations of Treprostinil or its derivative, or a pharmaceutically acceptable salt thereof, where the preparations may be isotonic with the blood of the intended recipient. These preparations may be administered by means of subcutaneous injection, although administration may also be effected intravenously or by means of intramuscular or intradermal injection. Such preparations may conveniently be prepared by admixing the compound with water or a glycine or citrate buffer and rendering the resulting solution sterile and isotonic with the blood. Injectable formulations according to the invention may contain from 0.1 to 5% w/v of active compound and may be administered at a rate of 0.1 ml/min/kg. Alternatively, the invention may administered at a rate of 0.625 to 50 ng/kg/min. Alternatively, the invention may be administered at a rate of 10 to 15 ng/kg/min.

Formulations suitable for oral administration may be presented in discrete units, such as capsules, cachets, lozenges, or tablets, each containing a predetermined amount of Treprostinil or its derivative, or a pharmaceutically acceptable salt thereof, as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients).

In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising Treprostinil or its derivative, or a pharmaceutically acceptable salt thereof, in a flavored base, usually sucrose and acacia or tragacanth; and pastilles comprising the compound in an inert base such as gelatin and glycerin or sucrose and acacia.

Formulations suitable for rectal administration are preferably presented as unit dose suppositories. These may be prepared by admixing Treprostinil or its derivative, or a pharmaceutically acceptable salt thereof, with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include vaseline, lanoline, polyethylene glycols, alcohols, and combinations of two or more thereof. The active compound is generally present at a concentration of from 0.1 to 15% w/w, for example, from 0.5 to 2% w/w. Formulations for transdermal administration may be delivered by iontophoresis (see, for example, Pharmaceutical Research, 3(6): 318 (1986)) and typically take the form of an optionally buffered aqueous solution of Treprostinil or its derivative or salt or thereof. Suitable formulations comprise citrate or bis/tris buffer (pH 6) or ethanol/water and contain from 0.1 to 0.2M active ingredient.

Treprostinil or its derivative or a pharmaceutically acceptable salt thereof can be conveniently prepared by methods the same as or analogous to those described in U.S. Pat. No. 4,306,075, U.S. Pat. No. 6,528,688 and U.S. Pat. No. 6,441,245.

Preferably, treprostinil is administered by inhalation, which in the present context refers to the delivery of the active ingredient or a combination of active ingredients through a respiratory passage, wherein the subject in need of the active ingredient(s) through the subjects airways, such as the nose or mouth.

Treprostinil can be administered by inhalation using an inhalation device such as a nebulizer, inhaler such as a metered dose inhaler, atomizer or aerosolizer. In some embodiments, the inhalation device can form droplets from a solution or liquid containing the active ingredients. The droplets can be less than 10 micrometers in diameter. One example of appropriate inhalation device is the AM-601 MEDICATOR AEROSOL DELIVERY SYSTEM™ (a nebulizer manufactured by Healthline Medical in Baldwin Park, Calif.).

In some embodiments, the inhalation devise can be a metered dose inhaler, which in the present context means a device capable of delivering a metered or bolus dose of respiratory drug, such as treprostinil, to the lungs. One example of the inhalation device can be a pressurized metered dose inhaler, a device which produces the aerosol clouds for inhalation from solutions of respiratory drugs in chlorofluorocarbon (CFC) and/or hydrofluoroalkane (HFA) solutions.

The inhalation device can be also a dry powder inhaler. In such case, the respiratory drug is inhaled in solid formulation, usually in the form of a powder with particle size less than 10 micrometers in diameter or less than 5 micrometers in diameter.

The metered dose inhaler can be a soft mist inhaler (SMI), in which the aerosol cloud can be generated by passing a solution containing a respiratory drug through a nozzle or series of nozzles. The aerosol generation can be achieved in SMI, for example, by mechanical, electromechanical or thermomechanical process. Examples of soft mist inhalers include the Respimat® Inhaler (Boeringer Ingelheim GmbH), the AERx® Inhaler (Aradigm Corp.), the Mystic™ Inhaler (Ventaira Pharmaceuticals, Inc) and the Aira™ Inhaler (Chrysalis Technologies Incorporated). For a review of soft mist inhaler technology, see e.g. M. Hindle, The Drug Delivery Companies Report, Autumn/Winter 2004, pp. 31-34. The aerosols for SMI can be generated from a solution of a respiratory drug containing pharmaceutically acceptable excipients. The solution can be, for example, a solution of treprostinil in water, ethanol or a mixture of the two.

The amount of treprostinil that can be administered in a single event using a metered dose inhaler can be from about 15 μg to about 100 μg or from about 15μg to about 90 μg or from about 30 μg to about 90 μg or from about 30 μg to about 6011g.

Administration of treprostinil using a metered dose inhaler can be carried out by administering a formulation with treprostinil concentration from about 500 μg/ml to about 2500 μg/ml, or from about 800 μg/ml to about 2200 μg/ml, or from about 1000 μg/ml to about 2000 μg/ml.

The Rho kinase inhibitor can be any compound that inhibits Rho kinase activity with efficiency of at least 40% or at least 60% or at least 80% or at least 90% or at least 95%. The efficiency of Rho kinase inhibition of a particular compound can be determined, for example, using Rho kinase biochemical essay as detailed in column 44 of U.S. Pat. No. 6,943,172 issued on Sep. 13, 2005, to Nagarathnam et. al. incorporated herein by reference in its entirety.

Rho kinase inhibitors such as fasudil, hydroxyfasudil and Y-27632 were suggested for treatment of pulmonary hypertension, see e.g. Abe K, Shimokawa H, Morikawa, et al. 2004. Long-term treatment with a Rho-kinase inhibitor improves monocrotaline-induced fatal pulmonary hypertension in rats. Circ Res. 94:385-93; Fukumoto Y, Matoba T, Ito A, et al. 2005. Acute vasodilator effects of a Rho-kinase inhibitor, fasudil, in patients with severe pulmonary hypertension. Heart. 91:391-92; Ishikura K, Yamada N, Ito M, et al. 2006. Beneficial acute effects of Rho-kinase inhibitor in patients with pulmonary arterial hypertension. Circ J. 70:174-78 ; Fagan K A, Oka M, Bauer N R, et al. 2004. Attenuation of acute hypoxic pulmonary vasoconstriction and hypoxic pulmonary hypertension in mice by inhibition of Rho-kinase. Am J Physiol Lung Cell Mol Physiol. 287:L656-64.

In some embodiments, the Rho kinase inhibitor can be a compound of the formula (I):

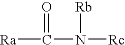

where Ra is a group of the formula

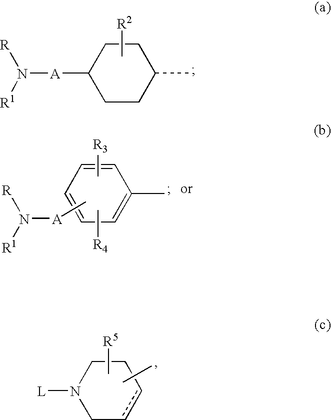

in the formulas (a) and (b), R is hydrogen, alkyl or cycloalkyl, cycloalkylalkyl, phenyl or aralkyl, which optionally have a substituent on the ring, or a group of the formula

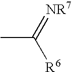

wherein R⁶ is hydrogen, alkyl or formula: —NR⁸R⁹, wherein R⁸ and R⁹ are the same or different and each is hydrogen, alkyl, aralkyl or phenyl, R⁷ is hydrogen, alkyl, aralkyl, phenyl, nitro or cyano, or R⁶ and R⁷ in combination show a group forming a heterocycle optionally having, in the ring, oxygen atom, sulfur atom or optionally substituted nitrogen atom, R¹ is hydrogen, alkyl or cycloalkyl, cycloalkylalkyl, phenyl or aralkyl, which optionally have a substituent on the ring, or R and R¹ in combination form, together with the adjacent nitrogen atom, a group forming a heterocycle optionally having, in the ring, oxygen atom, sulfur atom or optionally substituted nitrogen atom, R² is hydrogen or alkyl, R³ and R⁴ are the same or different and each is hydrogen, alkyl, aralkyl, halogen, nitro, amino, alkylamino, acylamino, hydroxy, alkoxy, aralkyloxy, cyano, acyl, mercapto, alkylthio, aralkylthio, carboxy, alkoxycarbonyl, carbamoyl, alkylcarbamoyl or azide, and A is a group of the formula

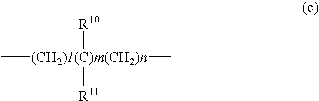

wherein R¹⁰ and R¹¹ are the same or different and each is hydrogen, alkyl, haloalkyl, aralkyl, hydroxyalkyl, carboxy or alkoxycarbonyl, or R¹⁰ and R¹¹ show a group, which forms cycloalkyl in combination and l, m and n are each 0 or an integer ranging from 1 to 3. In the formula (c), L is hydrogen, alkyl, aminoalkyl, mono or dialkylaminoalkyl, tetrahydrofurfuryl, carbamoylalkyl, phthalimidoalkyl, amidino or a group of the formula

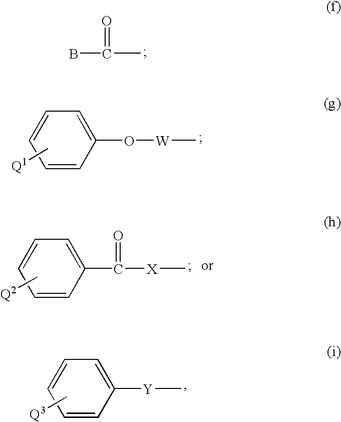

wherein B is hydrogen, alkyl, alkoxy, aralkyl, aralkyloxy, aminoalkyl, hydroxyalkyl, alkanoyloxyalkyl, alkoxycarbonylalkyl, alpha.-aminobenzyl, furyl, pyridyl, phenyl, phenylamino, styryl or imidazopyridyl, Q¹ is hydrogen, halogen, hydroxy, aralkyloxy or thienylmethyl, W is alkylene, Q² is hydrogen, halogen, hydroxy or aralkyloxy, X is alkylene, Q³ is hydrogen, halogen, hydroxy, alkoxy, nitro, amino, 2,3-dihydrofuryl or 5-methyl-3-oxo-2,3,4,5-tetrahydropyridazin-6-yl; and Y is a single bond, alkylene or alkenylene, and in the formula (c), a broken line is a single bond or a double bond, and R⁵ is hydrogen, hydroxy, alkoxy, alkoxycarbonyloxy, alkanoyloxy or aralkyloxycarbonyloxy; Rb is a hydrogen, an alkyl, an aralkyl, an aminoalkyl or a mono- or dialkylaminoalkyl; and Rc is an optionally substituted heterocycle containing nitrogen, an isomer thereof and/or a pharmaceutically acceptable acid addition salt thereof.

Compounds of the formula (I) are exemplified by the following compounds: (1) 4-(2-pyridylcarbamoyl)piperidine; (2)1-benzyloxycarbonyl-4-(4-pyridylcarbamoyl)piperidine; (3) 1-benzoyl-4-(4-pyridylcarbamoyl)piperidine; (4) 1-propyl-4-(4-pyridylcarbamoyl)piperidine; (5) 1-[3-(2-(2-thienylmethyl)phenoxy)-2-hydroxypropyl]-4-(4-pyridylcarbamoyl)-piperidine; (6) 4-(4-pyridylcarbamoyl)piperidine; (7) 1-benzyl-4-(4-pyridylcarbamoyl)-1,2,5,6-tetrahydropyridine; (8) 3-(4-pyridylcarbamoyl)piperidine; (9) 1-benzyl-3-(4-pyridylcarbamoyl)piperidine; (10) 1-(2-(4-benzyloxyphenoxy)ethyl)-4-(N-(2-pyridyl)-N-benzylcarbamoyl)pyridine; (11) 1-formyl-4-(4-pyridylcarbamoyl)piperidine; (12) 4-(3-pyridylcarbamoyl)piperidine; (13) 1-isopropyl-4-(4-pyridylcarbamoyl)piperidine; (14) 1-methyl-4-(4-pyridylcarbamoyl)piperidine; (15) 1-hexyl-4-(4-pyridylcarbamoyl)piperidine; (16) 1-benzyl-4-(4-pyridylcarbamoyl)piperidine; (17) 1-(2-phenylethyl)-4-(4-pyridylcarbamoyl)piperidine; (18) 1-(2-(4-methoxyphenyl)ethyl)-4-(4-pyridylcarbamoyl)piperidine; (19) 1-(2-(4-methoxyphenyl)ethyl)-4-(2-pyridylcarbamoyl)piperidine; (20) 1-(2-(4-chlorophenyl)ethyl)-4-(4-pyridylcarbamoyl)piperidine; (21) 1-diphenylmethyl-4-(2-pyridylcarbamoyl)piperidine; (22) 1-[2-(4-(5-methyl-3-oxo-2,3,4,5-tetrahydropyridazin-6-yl)phenyl)ethyl]-4-(2-pyridylcarbamoyl)piperidine; (23) 1-(4-(4,5-dihydro-2-furyl)phenyl)-4-(4-pyridylcarbamoyl)piperidine; (24) 1-(2-nitrophenyl)-4-(4-pyridylcarbamoyl)piperidine; (25) 1-(2-aminophenyl)-4-(4-pyridylcarbamoyl)piperidine; (26) 1-nicotinoyl-4-(4-pyridylcarbamoyl)piperidine; (27) 1-isonicotinoyl-4-(4-pyridylcarbamoyl)piperidine; (28) 1-(3,4,5-trimethoxybenzoyl)-4-(4-pyridylcarbamoyl)piperidine; (29) 1-acetyl-4-(4-pyridylcarbamoyl)piperidine; (30) 1-(3-(4-fluorobenzoyl)propyl)-4-(4-pyridylcarbamoyl)piperidine; (31) 1-(3-(4-fluorobenzoyl)propyl)-4-(2-pyridylcarbamoyl)piperidine; (32) 1-(1-(4-hydroxybenzoyl)ethyl)-4-(2-pyridylcarbamoyl)piperidine; (33) 1-(1-(4-benzyloxybenzoyl)ethyl)-4-(2-pyridylcarbamoyl)piperidine; (34) 1-(2-(4-hydroxyphenoxy)ethyl)-4-(2-pyridylcarbamoyl)piperidine; (35) 1-(4-(4-fluorophenyl)-4-hydroxybutyl)-4-(4-pyridylcarbamoyl)piperidine; (36) 1-(1-methyl-2-(4-hydroxyphenyl)-2-hydroxyethyl)-4-(2-pyridylcarbamoyl)pipe ridine; (37) 1-cinnamyl-4-(2-pyridylcarbamoyl)piperidine; (38) 1-(2-hydroxy-3-phenoxypropyl)-4-(4-pyridylcarbamoyl)piperidine; (39) 1-(2-hydroxy-3-phenoxypropyl)-4-(3-pyridylcarbamoyl)piperidine; (40) 1-(2-hydroxy-3-phenoxypropyl)-4-(2-pyridylcarbamoyl)piperidine; (41) 1-(2-phenylethyl)-4-[N-(2-pyridyl)-N-(2-(N,N-dimethylamino)ethyl)carbamoyl]piperidine; (42) 1-benzyloxycarbonyl-4-(2-pyridylcarbamoyl)piperidine; (43) 1-(3-chlorophenyl)carbamoyl-4-(4-pyridylcarbamoyl)piperidine; (44) 4-[N-(2-pyridyl)-N-(2-(N,N-dimethylamino)ethyl)carbamoyl]piperidine; (45) 1-methyl-4-(4-pyridylcarbamoyl)-1,2,5,6-tetrahydropyridine; (46) 1-nicotinoyl-3-(4-pyridylcarbamoyl)piperidine; (47) 1-[2-(4-fluorobenzoyl)ethyl]-4-(4-pyridylcarbamoyl)piperidine; (48) 1-(6-chloro-2-methylimidazo[1,2-a]pyridine-3-carbonyl)-4-(4-pyridylcarbamoyl)piperidine; (49) 1-(4-nitrobenzyl)-4-(4-pyridylcarbamoyl)piperidine; (50) 1-hexyl-4-(4-pyridylcarbamoyl)piperidine; (51) 1-benzyloxycarbonyl-4-(2-chloro-4-pyridylcarbamoyl)piperidine; (52) 4-(2-chloro-4-pyridylcarbamoyl)piperidine; (53) 1-(2-chloronicotinoyl)-4-(4-pyridylcarbamoyl)piperidine; (54) 3-(2-chloro-4-pyridylcarbamoyl)piperidine; (55) 1-(4-phthalimidobutyl)-4-(4-pyridylcarbamoyl)piperidine; (56) 1-(3,5-di-tert-butyl-4-hydroxycinnamoyl)-4-(4-pyridylcarbamoyl)piperidine; (57) 1-carbamoylmethyl-4-(4-pyridylcarbamoyl)piperidine; (58) 1-benzyloxycarbonyl-4-(5-nitro-2-pyridylcarbamoyl)piperidine; (59) 4-(5-nitro-2-pyridylcarbamoyl)piperidine; (60) trans-4-benzyloxycarboxamidomethyl-1-(4-pyridylcarbamoyl)cyclohexane; (61) trans-4-aminomethyl-1-(4-pyridylcarbamoyl)cyclohexane; (62) trans-4-formamidomethyl-1-(4-pyridylcarbamoyl)cyclohexane; (63) trans-4-dimethylaminomethyl-1-(4-pyridylcarbamoyl)cyclohexane; (64) N-benzylidene-trans-(4-pyridylcarbamoyl)cyclohexylmethylamine; (65) trans-4-benzylaminomethyl-1-(4-pyridylcarbamoyl)cyclohexane; (66) trans-4-isopropylaminomethyl-1-(4-pyridylcarbamoyl)cyclohexane; (67) trans-4-nicotinoylaminomethyl-1-(4-pyridylcarbamoyl)cyclohexane; (68) trans-4-cyclohexylaminomethyl-1-(4-pyridylcarbamoyl)cyclohexane; (69) trans-4-benzyloxycarboxamide-1-(4-pyridylcarbamoyl)cyclohexane; (70) trans-4-amino-1-(4-pyridylcarbamoyl)cyclohexane; (71) trans-4-(1-aminoethyl)-1-(4-pyridylcarbamoyl)cyclohexane; (72) trans-4-aminomethyl-cis-2-methyl-1-(4-pyridylcarbamoyl)cyclohexane; (73) (+)-trans-4-(1-benzyloxycarboxamidopropyl)-1-cyclohexanecarboxylic acid; (74) (+)-trans-4-(1-benzyloxycarboxamidopropyl)-1-(4-pyridylcarbamoyl)cyclohexane; (75) (−)-trans-4-(1-benzyloxycarboxamidpropyl)-1-(4-pyridylcarbamoyl)cyclohexane; (76) (+)-trans-4-(1-aminopropyl)-1-(4-pyridylcarbamoyl)cyclohexane; (77) (−)-trans-4-(1-aminopropyl)-1-(4-pyridylcarbamoyl)cyclohexane; (78) (−)-trans-4-(1-benzyloxycarboxamidoethyl)-1-(4-pyridylcarbamoyl)cyclohexane; (79) (+)-trans-4-(1-benzyloxycarboxamidoethyl)-1-(4-pyridylcarbamoyl)cyclohexane; (80) (+)-trans-4-(1-aminoethyl)-1-(4-pyridylcarbamoyl)cyclohexane; (81) (−)-trans-4-(1-aminoethyl)-1-(4-pyridylcarbamoyl)cyclohexane; (82) trans-4-(4-chlorobenzoyl)aminomethyl-1-(4-pyridylcarbamoyl)cyclohexane; (83) trans-4-aminomethyl-1-(2-pyridylcarbamoyl)cyclohexane; (84) trans-4-benzyloxycarboxamidomethyl-1-(2-pyridylcarbamoyl)cyclohexane; (85) trans-4-methylaminomethyl-1-(4-pyridylcarbamoyl)cyclohexane; (86) trans-4-(N-benzyl-N-methylamino)methyl-1-(4-pyridylcarbamoyl)cyclohexane; (87) trans-4-aminomethyl-1-(3-pyridylcarbamoyl)cyclohexane; (88) trans-4-aminomethyl-1-[(3-hydroxy-2-pyridyl)carbamoyl]cyclohexane; (89) trans-4-benzyloxycarboxamidomethyl-1-(3-pyridylcarbamoyl)cyclohexane; (90) trans-4-benzyloxycarboxamidomethyl-1-[(3-benzyloxy-2-pyridyl)carbamoyl]cyclohexane; (91) trans-4-phthalimidomethyl-1-(4-pyridylcarbamoyl)cyclohexane; (92) trans-4-benzyloxycarboxamidomethyl-1-(3-methyl-4-pyridylcarbamoyl)cyclohexane; (93) trans-4-aminomethyl-1-(3-methyl-4-pyridylcarbamoyl)cyclohexane; (94) 4-(trans-4-benzyloxycarboxamidomethylcyclohexylcarbonyl)amino-2,6-dimethyl pyridine-N-oxide; (95) 4-(trans-4-aminomethylcyclohexylcarbonyl)amino-2,6-dimethylpyridine-N-oxide; (96) trans-4-aminomethyl-1-(2-methyl-4-pyridylcarbamoyl)cyclohexane; (97) trans-4-(1-benzyloxycarboxamidoethyl)-1-(4-pyridylcarbamoyl)cyclohexane; (98) trans-4-(1-amino-1-methylethyl)-1-(4-pyridylcarbamoyl)cyclohexane; (99) trans-4-(2-aminoethyl)-1-(4-pyridylcarbamoyl)cyclohexane; (100) trans-4-(2-amino-1-methylethyl)-1-(4-pyridylcarbamoyl)cyclohexane; (101) trans-4-(1-aminopropyl)-1-(4-pyridylcarbamoyl)cyclohexane; (102) trans-4-aminomethyl-trans-1-methyl-1-(4-pyridylcarbamoyl)cyclohexane; (103) trans-4-benzylaminomethyl-cis-2-methyl-1-(4-pyridylcarbamoyl)cyclohexane; (104) trans-4-(1-benzyloxycarboxamide-1-methylethyl)-1-(4-pyridylcarbamoyl)-cyclohexane; (105) trans-4-benzyloxycarboxamidomethyl-1-(N-methyl-4-pyridylcarbamoyl)cyclohexane; (106) trans-4-(1-acetamide-1-methylethyl)-1-(4-pyridylcarbamoyl)cyclohexane; (107) trans-N-(6-amino-4-pyrimidyl)-4-aminomethylcyclohexanecarboxamide; (108) trans-N-(1H-pyrrolo[2,3-b]pyridin-4-yl)-4-aminomethylcyclohexanecarboxamide; (109) (+)-trans-N-(1H-pyrrolo[2,3-b]pyridin-4-yl)-4-(1-aminoethyl)cyclohexanecarboxamide; (110) trans-N-(1H-pyrrolo[2,3-b]pyridin-4-yl)-4-(11-amino-1-methylethyl)cyclohexanecarboxamide; (111) trans-N-(1H-pyrazolo [3,4-b]pyridin-4-yl)-4-aminomethylcyclohexanecarboxamide; (112) (+)-trans-N-(1H-pyrazolo[3,4-b]pyridin-4-yl)-4-(1-aminoethyl)cyclohexanecarboxamide; (113) trans-N-(1H-pyrazolo[3,4-b]pyridin-4-yl)-4-(1-amino-1-methylethyl)cyclohexanecarboxamide; (114) (+)-trans-N-(2-amino-4-pyridyl)-4-(1-aminoethyl)cyclohexanecarboxamide; (115) trans-N-(1H-pyrazolo[3,4-d]pyrimidin-4-yl)-4-aminomethylcyclohexanecarboxamide; (116) (+)-trans-N-(1H-pyrazolo[4-d]pyrimidin-4-yl)-4-(1-aminoethyl)cyclohexanecarboxamide; (117) trans-N-(1H-pyrazolo[3,4-d]pyrimidin-4-yl)-4-(1-amino-1-methylethyl)cyclohexanecarboxamide; (118) trans-N-(4-pyrimidinyl)-4-aminomethylcyclohexanecarboxamide; (119) trans-N-(3-amino-4-pyridyl)-4-aminomethylcyclohexanecarboxamide; (120) trans-N-(7H-imidazo[4,5-d]pyrimidin-6-yl)-4-aminomethylcyclohexanecarboxamide; (121) trans-N-(3H-1,2,3-triazolo[4,5-d]pyrimidin-7-yl)-4-aminomethylcyclohexanecarboxamide; (122) trans-N-(1-benzyl-1H-pyrazolo[3,4-b]pyridin-4-yl)-4-aminomethylcyclohexanecarboxamide; (123) trans-N-(1H-5-pyrazolyl)-4-aminomethylcyclohexanecarboxamide; (124) trans-N-(1H-pyrazolo[3,4-b]pyridin-4-yl)-4-aminomethylcyclohexanecarboxamide; (125) trans-N-(4-pyridazinyl)-4-aminomethylcyclohexanecarboxamide; (126) trans-N-(7H-pyrrolo[2,3-d]pyrimidin-4-yl)-4-aminomethylcyclohexanecarboxamide; (127) trans-N-(2-amino-4-pyridyl)-4-aminomethylcyclohexanecarboxamide; (128) trans-N-(thieno[2,3-d]pyrimidin-4-yl)-4-aminomethylcyclohexanecarboxamide; (129) trans-N-(5-methyl-1,2,4-triazolo[1,5-a]pyrimidin-7-yl)-4-aminomethylcyclohexanecarboxamide; (130) trans-N-(3-cyano-5-methylpyrazolo[1,5-a]pyrimidin-7-yl)-4-aminomethylcyclo hexanecarboxamide; (131) trans-N-(1 H-pyrazolo[3,4-b]pyridin-4-yl)-4-(1-amino-1-methylethyl)cyclohexanecarboxamide; (132) trans-N-(2-(1-pyrrolidinyl)-4-pyridyl)-4-aminomethylcyclohexanecarboxamide; (133) trans-N-(2,6-diamino-4-pyrimidyl)-4-aminomethylcyclohexanecarboxamide; (134) (+)-trans-N-(7-methyl-1,8-naphthyridin-4-yl)-4-(1-aminoethyl)cyclohexanecarboxamide; (135) trans-N-(1-benzyloxymethylpyrrolo[2,3-b]pyridin-4-yl)-4-aminomethylcyclohexanecarboxamide; (136) (+)-trans-N-(1-methylpyrrolo[2,3-b]pyridin-4-yl)-4-(1-aminoethyl)cyclohexanecarboxamide; (137) trans-N-benzyl-N-(2-benzylamino-4-pyridyl)-4-(1-amino-1-methylethyl)cyclohexanecarboxamide; (138) trans-N-(2-azide-4-pyridyl)-4-aminomethylcyclohexanecarboxamide; (139) trans-N-(2,3-dihydro-1H-pyrrolo[2,3-b]pyridin-4-yl)-4-aminomethylcyclohexanecarboxamide; (140) trans-N-(2,3-dihydro-1H-pyrrolo[2,3-b]pyridin-4-yl)-4-(1-amino-1-methylethyl)cyclohexanecarboxamide; (141-1) trans-N-(2-carboxy-4-pyridyl)-4-aminomethylcyclohexanecarboxamide; (141-2) (R)-(+)-trans-N-(3-bromo-1H-pyrrolo[2,3-b]pyridin-4-yl)-4-(1-aminoethyl)cyclohexanecarboxamide; (142) trans-N-(1H-pyrrolo[2,3-b]pyridin-4-yl)-4-guanidinomethylcyclohexanecarboxamide; (143) trans-N-(1H-pyrazolo[3,4-b]pyridin-4-yl)-4-guanidinomethylcyclohexanecarboxamide; (144) trans-N-(4-pyridyl)-4-guanidinomethylcyclohexanecarboxamide; (145) trans-N-(1-methylpyrrolo[2,3-b]pyridin-4-yl)-4-(guanidinomethyl)cyclohexanecarboxamide; (146) trans-N-(1H-pyrrolo[2,3-b]pyridin-4-yl)-4-(2-imidazolin-2-yl)aminomethylcyclohexanecarboxamide; (147) trans-N-(1-benzyloxymethylpyrrolo[2,3-b]pyridin-4-yl)-4-guanidinomethylcyclohexanecarboxamide; (148) trans-N-(2-amino-4-pyridyl)-4-guanidinomethylcyclohexanecarboxamide; (149) trans-N-(1-benzyloxymethyl-1H-pyrrolo[2,3-b]pyridin-4-yl)-4-(2-imidazolin-2-yl)aminomethylcyclohexanecarboxamide; (150) trans-N-(1H-pyrrolo[2,3-b]pyridin-4-yl)-4-(3-benzylguanidinomethyl)-cyclohexanecarboxamide; (151) trans-N-(1H-pyrrolo[2,3-b]pyridin-4-yl)-4-(3-phenylguanidinomethyl)cyclohexanecarboxamide; (152) trans-N-(1H-pyrrolo[2,3-b]pyridin-4-yl)-4-(3-propylguanidinomethyl)cyclohexanecarboxamide; (153) trans-N-(1H-pyrrolo[2,3-b]pyridin-4-yl)-4-(3-octylguanidinomethyl)cyclohexanecarboxamide; (154) trans-N-(1-benzyloxymethylpyrrolo[2,3-b]pyridin-4-yl)-4-(2-benzyl-3-ethylguanidinomethyl)cyclohexanecarboxamide; (155) trans-N-(1H-pyrrolo[2,3-b]pyridin-4-yl)-4-(imidazol-2-yl)aminomethylcyclohexanecarboxamide; (156) trans-N-(1H-pyrrolo[2,3-b]pyridin-4-yl)-4-(thiazol-2-yl)aminomethylcyclohexanecarboxamide; (157) (R)-(+)-N-(4-pyridyl)-4-(1-aminoethyl)benzamide; (158) N-(4-pyridyl)-4-(1-amino-1-methylethyl)benzamide; (159) N-(4-pyridyl)-4-aminomethyl-2-benzyloxybenzamide; (160) N-(4-pyridyl)-4-aminomethyl-2-ethoxybenzamide; (161) (R)-(−)-N-(4-pyridyl)-4-(1-aminoethyl)-3-nitrobenzamide; (162) (R)-(−)-N-(4-pyridyl)-3-amino-4-(1-aminoethyl)benzamide; (163) (R)-(+)-N-(4-pyridyl)-4-(1-aminoethyl)-3-chlorobenzamide; (164) N-(4-pyridyl)-3-aminomethylbenzamide; (165) (R)-(+)-N-(1H-pyrrolo[2,3-b]pyridin-4-yl)-4-(1-aminoethyl)benzamide; (166) (R)-(+)-N-(1H-pyrazolo[3,4-b]pyridin-4-yl)-4-(1-aminoethyl)benzamide; (167) N-(1H-pyrazolo[3,4-b]pyridin-4-yl)-4-guanidinomethylbenzamide; (168) N-(4-pyridyl)-4-guanidinomethylbenzamide; (169) (R)-(+)-N-(4-pyridyl)-4-(1-aminoethyl)-3-fluorobenzamide; (170) N-(4-pyridyl)-4-aminomethylbenzamide; (171) N-(4-pyridyl)-4-aminomethyl-2-hydroxybenzamide; (172) N-(4-pyridyl)-4-(2-aminoethyl)benzamide; (173) N-(4-pyridyl)-4-aminomethyl-3-nitrobenzamide; (174) N-(4-pyridyl)-3-amino-4-aminomethylbenzamide; (175) (S)-(−)-N-(4-pyridyl)-4-(1-aminoethyl)benzamide; (176) (S)-(−)-N-(4-pyridyl)-2-(1-aminoethyl)benzamide; (177) (R)-(+)-N-(4-pyridyl)-4-(1-aminoethyl)-2-chlorobenzamide; (178) (R)-(+)-N-(1H-pyrrolo[2,3-b]pyridin-4-yl)-4-(1-(3-propylguanidino)ethyl)benzamide; (179) (R)-(−)-N-(1H-pyrrolo[2,3-b]pyridin-4-yl)-4-(1-aminoethyl)-3-azidebenzamide; (180) (R)-(+)-N-(4-pyridyl)-4-(1-aminoethyl)-2-nitrobenzamide; (181) (R)-(−)-N-(4-pyridyl)-4-(1-aminoethyl)-3-ethoxybenzamide; (182) (R)-(+)-N-(3-iodo-1H-pyrrolo[2,3-b]pyridin-4-yl)-4-(1-aminoethyl)benzamide; (183) (R)-(+)-N-(3-iodo-1H-pyrrolo[2,3-b]pyridin-4-yl)-4-(1-aminoethyl)-3-azidebenzamide; (184) (R)-(−)-N-(4-pyridyl)-4-(1-aminoethyl)-3-hydroxybenzamide; (185) N-(1H-pyrazolo[3,4-b]pyridin-4-yl)-4-guanidinomethyl-3-nitrobenzamide; (186) (R)-N-(1H-pyrazolo[3,4-b]pyridin-4-yl)-4-(1-guanidinoethyl)-3-nitrobenzamide; (187) (R)-N-(1H-pyrazolo[4-b]pyridin-4-yl)-4-(1-aminoethyl)-2-nitrobenzamide; (188) N-(1H-pyrazolo [,4-b]pyridin-4-yl)-4-guanidinobenzamide; (189) (R)-N-(1H-pyrazolo [3,4-b]pyridin-4-yl)-4-(1-aminoethyl)-3-nitrobenzamide; (190) (R)-N-(1H-pyrazolo[4-b]pyridin-4-yl)-4-(1-guanidinoethyl)benzamide; (191) N-(1H-pyrazolo[3,4-b]pyridin-4-yl)-4-(1-amino-2-hydroxyethyl)benzamide; (192) N-(1H-pyrazolo[3,4-b]pyridin-4-yl)-4-aminomethyl-3-nitrobenzamide; (193) N-(1H-pyrrolo[2,3-b]pyridin-4-yl)-4-piperidinecarboxamide; (194) N-(1H-pyrazolo[3,4-b]pyridin-4-yl)-4-piperidinecarboxamide; (195) N-(1H-pyrazolo[3,4-b]pyridin-4-yl)-1-aminoacetyl-4-piperidinecarboxamide; (196) N-(1-methoxymethyl-1H-pyrazolo[3,4-b]pyridin-4-yl)-4-piperidinecarboxamide; (197) N-(2,3-dihydro-1H-pyrrolo[2,3-b]pyridin-4-yl)-4-piperidinecarboxamide; (198) N-(1H-pyrrolo[2,3-b]pyridin-4-yl)-1-(2-phenylethyl)-4-piperidinecarboxamide; (199) N-(1H-pyrrolo[2,3-b]pyridin-4-yl)-1-amidino-4-piperidinecarboxamide; (200) N-(1H-pyrrolo [2,3-b]pyridin-4-yl)-1-(3-phenylpropyl)-4-piperidinecarboxamide; (201) N-(1H-pyrrolo[2,3-b]pyridin-4-yl)-1-benzyl-4-piperidinecarboxamide; (202) N-(1H-pyrazolo[3,4-b]pyridin-4-yl)-1-(2-phenylethyl)-4-piperidinecarboxamide; (203) N-(1H-pyrazolo [3,4-b]pyridin-4-yl)-1-(3-phenylpropyl)-4-piperidinecarboxamide. Preferred are compounds (80), (109), (110), (112), (115), (142), (143), (144), (145), (153), (157), (163), (165), (166) and (179).

In some embodiments, the Rho kinase inhibitor can comprise a compound of the formula (II):

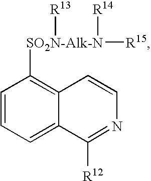

wherein R¹² is a hydrogen, a chlorine or a hydroxy, and when R¹² is a hydrogen, Alk is an alkylene having 2 to 6 carbon atoms, which optionally has alkyl having 1 to 10 carbon atoms, aryl or aralkyl as a substituent; R¹³ is a hydrogen; R¹⁴ is a hydrogen, or a linear or branched alkyl having 1 to 6 carbon atoms, an aryl or an aralkyl; R¹⁵ is a hydrogen, a linear or branched alkyl having 1 to 6 carbon atoms, an aryl or an aralkyl, or a benzoyl, a cinnamyl, a cinnamoyl, a furoyl or a group of the following formula

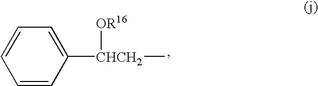

wherein R¹⁶ is linear or branched alkyl having 1 to 6 carbon atoms or a group of the following formula

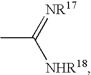

wherein R¹⁷ and R¹⁸ are hydrogen or directly bonded to form alkylene having 2 to 4 carbon atoms; or R¹³ and R¹⁴ are directly bonded to form alkylene having 4 or less carbon atoms, which is optionally substituted by alkyl having 1 to 10 carbon atoms, phenyl or benzyl, or R¹⁴ and R¹⁵ directly or in combination via oxygen atom form a heterocycle together with the adjacent nitrogen atom, and when R¹² is a chlorine or a hydroxy, Alk is an alkylene having 2 to 6 carbon atoms, which is optionally substituted at the hydrogen bonded to carbon by alkyl having 1 to 6 carbon atoms, R¹³ and R¹⁴ are each a hydrogen, a linear or branched alkyl having 1 to 6 carbon atoms or directly bonded to each other to form ethylene or trimethylene, wherein hydrogen bonded to carbon is optionally substituted by alkyl having 1 to 6 carbon atoms; or R¹⁵ is a hydrogen, a linear or branched alkyl having 1 to 6 carbon atoms or an amidino, an isomer thereof and/or a pharmaceutically acceptable acid addition salt thereof.

Compounds of the formula (II) are exemplified by the following compounds: (204) 1-(5-isoquinolinesulfonyl)homopiperazine; (205) 1-(5-isoquinolinesulfonyl)-2-methylhomopiperazine; (206) 1-(5-isoquinolinesulfonyl)-3-methylhomopiperazine; (207) 1-(5-isoquinolinesulfonyl)-6-methylhomopiperazine; (208) 1-(5-isoquinolinesulfonyl)-2,3-dimethylhomopiperazine; (209) 1-(5-isoquinolinesulfonyl)-3,3-dimethylhomopiperazine; (210) 1-(5-isoquinolinesulfonyl)-3-ethylhomopiperazine; (211) 1-(5-isoquinolinesulfonyl)-3-propylhomopiperazine; (212) 1-(5-isoquinolinesulfonyl)-3-isobutylhomopiperazine; (213) 1-(5-isoquinolinesulfonyl)-3-phenylhomopiperazine; (214) 1-(5-isoquinolinesulfonyl)-3-benzylhomopiperazine; (215) 1-(5-isoquinolinesulfonyl)-6-ethylhomopiperazine; (216) 1-(5-isoquinolinesulfonyl)-6-propylhomopiperazine; (217) 1-(5-isoquinolinesulfonyl)-6-butylhomopiperazine; (218) 1-(5-isoquinolinesulfonyl)-6-pentylhomopiperazine; (219) 1-(5-isoquinolinesulfonyl)-6-hexylhomopiperazine; (220) 1-(5-isoquinolinesulfonyl)-6-phenylhomopiperazine; (221) 1-(5-isoquinolinesulfonyl)-6-benzylhomopiperazine; (222) 1-(5-isoquinolinesulfonyl)-4-methylhomopiperazine; (223) 1-(5-isoquinolinesulfonyl)-4-ethylhomopiperazine; (224) 1-(5-isoquinolinesulfonyl)-4-propylhomopiperazine; (225) 1-(5-isoquinolinesulfonyl)-4-butylhomopiperazine; (226) 1-(5-isoquinolinesulfonyl)-4-hexylhomopiperazine; (227) N-(2-aminoethyl)-1-chloro-5-isoquinolinesulfonamide; (228) N-(4-aminoethyl)-1-chloro-5-isoquinolinesulfonamide; (229) N-(2-amino-1-methylethyl)-1-chloro-5-isoquinolinesulfonamide; (230) N-(2-amino-1-methylpentyl)-1-chloro-5-isoquinoline; (231) N-(3-amino-2-methylbutyl)-1-chloro-5-isoquinolinesulfonamide; (232) N-(3-di-n-butylaminopropyl)-1-chloro-5-isoquinolinesulfonamide; (233) N-(N-cyclohexyl-N-methylaminoethyl)-1-chloro-5-isoquinolinesulfonamide; (234) N-(2-guanidinoethyl)-1-chloro-5-isoquinolinesulfonamide; (235) N-(2-guanidinobutyl)-1-chloro-5-isoquinolinesulfonamide; (236) N-(2-guanidino-1-methylethyl)-1-chloro-5-isoquinolinesulfonamide; (237) N-(2-guanidinomethylpentyl)-1-chloro-5-isoquinolinesulfonamide; (238) N-(2-guanidino-3-methylbutyl)-1-chloro-5-isoquinolinesulfonamide; (239) N-(3-guanidino-2-methylpropyl)-1-chloro-5-isoquinolinesulfonamide; (240) N-(4-guanidino-3-methylbutyl)-1-chloro-5-isoquinolinesulfonamide; (241) 2-methyl-4-(1-chloro-5-isoquinolinesulfonyl)piperazine; (242) 2-ethyl-4-(1-chloro-5-isoquinolinesulfonyl)piperazine; (243) 2-isobutyl-4-(1-chloro-5-isoquinolinesulfonyl)piperazine; (244) 2,5-dimethyl-4-(1-chloro-5-isoquinolinesulfonyl)piperazine; (245) 1-methyl-4-(1-chloro-5-isoquinolinesulfonyl)piperazine; (246) 1-amidino-4-(1-chloro-5-isoquinolinesulfonyl)piperazine; (247) 1-amidino-4-(1-chloro-5-isoquinolinesulfonyl)homopiperazine; (248) 1-amidino-3-methyl-4-(1-chloro-5-isoquinolinesulfonyl)piperazine; (249) 1-amidino-2,5-dimethyl-4-(1-chloro-5-isoquinolinesulfonyl)piperazine; (250) N-(2-aminoethyl)-1-hydroxy-5-isoquinolinesulfonamide; (251) N-(4-aminobutyl)-1-hydroxy-5-isoquinolinesulfonamide; (252) N-(2-amino-1-methylethyl)-1-hydroxy-5-isoquinolinesulfonamide; (253) N-(2-amino-1-methylheptyl)-1-hydroxy-5-isoquinolinesulfonamide; (254) N-(3-amino-2-methylbutyl)-1-hydroxy-5-isoquinolinesulfonamide; (255) N-[3-(N,N-dibutylamino)propyl]-1-hydroxy-5-isoquinolinesulfonamide; (256) N-[2-(N-cyclohexyl-N-methylamino)ethyl]-1-hydroxy-5-isoquinolinesulfonamide; (257) N-(2-guanidinoethyl)-1-hydroxy-5-isoquinolinesulfonamide; (258) N-(4-guanidinobutyl)-1-hydroxy-5-isoquinolinesulfonamide; (259) N-(2-guanidino-1-methylethyl)-1-hydroxy-5-isoquinolinesulfonamide; (260) N-(1-guanidinomethylpentyl)-1-hydroxy-5-isoquinolinesulfonamide; (261) N-(2-guanidino-3-methylbutyl)-1-hydroxy-5-isoquinolinesulfonamide; (262) N-(3-guanidino-2-methylpropyl)-1-hydroxy-5-isoquinolinesulfonamide; (263) N-(4-guanidino-3-methylbutyl)-1-hydroxy-5-isoquinolinesulfonamide; (264) 2-methyl-4-(1-hydroxy-5-isoquinolinesulfonyl)piperazine; (265) 2-ethyl-4-(1-hydroxy-5-isoquinolinesulfonyl)piperazine; (266) 2-isobutyl-4-(1-hydroxy-5-isoquinolinesulfonyl)piperazine; (267) 2,5-dimethyl-4-(1-hydroxy-5-isoquinolinesulfonyl)piperazine; (268) 1-methyl-4-(1-hydroxy-5-isoquinolinesulfonyl)piperazine; (269) 1-amidino-4-(1-hydroxy-5-isoquinolinesulfonyl)piperazine; (270) 1-amidino-4-(1-hydroxy-5-isoquinolinesulfonyl)homopiperazine; (271) 1-amidino-3-methyl-4-(1-hydroxy-5-isoquinolinesulfonyl)piperazine; (272) 1-amidino-2,5-dimethyl-4-(1-hydroxy-5-isoquinolinesulfonyl)piperazine; (273) N-(2-methylaminoethyl)-1-chloro-5-isoquinolinesulfonamide; (274) N-(2-ethylaminoethyl)-1-chloro-5-isoquinolinesulfonamide; (275) N-(2-propylaminoethyl)-1-chloro-5-isoquinolinesulfonamide; (276) N-(2-butylaminoethyl)-1-chloro-5-isoquinolinesulfonamide; (277) N-(2-hexylaminoethyl)-1-chloro-5-isoquinolinesulfonamide; (278) 1-(1-chloro-5-isoquinolinesulfonyl)piperazine; (279) 1-(1-chloro-5-isoquinolinesulfonyl)homopiperazine; (280) N-(2-methylaminoethyl)-1-hydroxy-5-isoquinolinesulfonamide; (281) N-(2-ethylaminoethyl)-1-hydroxy-5-isoquinolinesulfonamide; (282) N-(2-propylaminoethyl)-1-hydroxy-5-isoquinolinesulfonamide; (283) N-(2-butylaminoethyl)-1-hydroxy-5-isoquinolinesulfonamide; (284) N-(2-hexylaminoethyl)-1-hydroxy-5-isoquinolinesulfonamide; (285) 1-(1-hydroxy-5-isoquinolinesulfonyl)piperazine; (286) 1-(1-hydroxy-5-isoquinolinesulfonyl)homopiperazine; (287) 1-(5-isoquinolinesulfonyl)-4-methylpiperazine; (288) 1-(5-isoquinolinesulfonyl)-4-n-hexylpiperazine; (289) 1-(5-isoquinolinesulfonyl)-4-cinnamylpiperazine; (290) 1-(5-isoquinolinesulfonyl)piperazine; (291) N-(2-aminoethyl)-5-isoquinolinesulfonamide; (292) N-(4-aminobutyl)-5-isoquinolinesulfonamide; (293) N-(3-di-n-butylaminopropyl)-5-isoquinolinesulfonamide; (294) 1-(5-isoquinolinesulfonyl)-3-methylpiperazine; (295) 1-(5-isoquinolinesulfonyl)-3-isobutylpiperazine; (296) 1-(5-isoquinolinesulfonyl)-2,5-dimethylpiperaine; (297) N-(3-guanidino-2-phenylpropyl)-5-isoquinolinesulfonamide; (298) N-(6-guanidino-1-methylheptyl)-5-isoquinolinesulfonamide; (299) 2-[2-(5-isoquinolinesulfonamide)ethylamino]-2-imidazoline; (300) 2-amidino-1-(5-isoquinolinesulfonyl)piperazine; (301) 4-amidino-2,5-dimethyl-1-(5-isoquinolinesulfonyl)piperazine; (302) 4-amidino-1-(5-isoquinolinesulfonyl)homopiperazine; (303) 4-(N1,N2-dimethylamidino)-1-(5-isoquinolinesulfonyl)piperazine; (304) 4-amidino-3-butyl-1-(5-isoquinolinesulfonyl)piperazine; (305) 4-hexyl-1-(5-isoquinolinesulfonyl)ethylenediamine; (306) N-(4-guanidinobutyl)-5-isoquinolinesulfonamide; (307) N-(2-guanidinoethyl)-5-isoquinolinesulfonamide; (308) 1-(5-isoquinolinesulfonyl)-2-methylpiperazine. Preferred are compounds (204) and (308).

The compound of the formulas (I) and (II) are disclosed in U.S. Pat. Nos. 4,997,834; 6,218,410; 6,451,825; and 6,906,061 incorporated herein by reference in their entirety.

The compound of the formula (I) can be synthesized according to the method disclosed in Japanese Patent Unexamined Publication No. 62-89679, Japanese Patent Unexamined Publication No. 3-218356, Japanese Patent Unexamined Publication No. 5-194401, Japanese Patent Unexamined Publication No. 6-41080, WO95/28387 and the like.

The compound of the formula (II) can be synthesized according to the method disclosed in Japanese Patent Unexamined Publication No. 57-156463, Japanese Patent Unexamined Publication No. 57-200366, Japanese Patent Unexamined Publication No. 58-121278, Japanese Patent Unexamined Publication No. 58-121279, Japanese Patent Unexamined Publication No. 59-93054, Japanese Patent Unexamined Publication No. 60-81168, Japanese Patent Unexamined Publication No. 61-152658, Japanese Patent Unexamined Publication No. 61-227581, Japanese Patent Unexamined Publication No. 62-103066, U.S. Pat. No. 4,678,783 and the like.

In some embodiments, the Rho kinase inhibitor can comprise a compound of the formula (III):

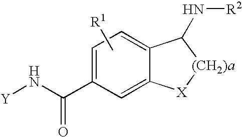

wherein R¹ is hydrogen, alkyl, cycloalkyl, halogen, hydroxyl, alkoxy, haloalkyl, hydroxyalkyl, aralkyl, acyl, alkoxycarbonyl, alkylcarbamoyl, alkylsulfone, nitro, amino optionally having substituents, cyano or phenyl; R² is hydrogen, alkyl, cycloalkyl, phenyl or aralkyl, or a group represented by the formula

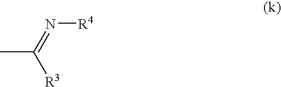

in the formula (k), R³ is hydrogen, alkyl or amino optionally having substituents, and R⁴ is hydrogen, alkyl, aralkyl, phenyl, nitro or cyano, or R³ and R⁴ may be bonded to form a heterocyclic ring containing, in the ring, oxygen atom, sulfur atom or nitrogen atom optionally having a substituent; a is an integer of 1 to 4; X is CH₂, O, S, SO₂ or NR⁷ wherein R⁷ is hydrogen, alkyl, aralkyl, haloalkyl or acyl; and Y is a group of the formula (l), (m), (n) or (o)

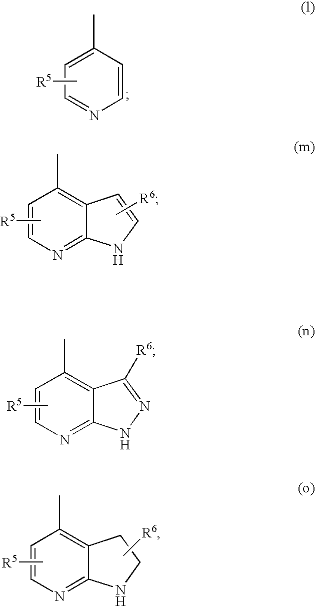

in the formulas (l), (m), (n) and (o), R⁵ and R⁶ are the same or different and each is hydrogen, alkyl, cycloalkyl, phenyl, halogen, hydroxyl, alkoxy, alkoxyalkyl, nitro, amino optionally having substituents or cyano, an isomer thereof or a pharmaceutically acceptable salt thereof.

Compounds of the formula (III) are exemplified by the following compounds: (RS)-4-amino-N-(4-pyridyl)thiochromane-7-carboxamide 1,1-dioxide; (RS)-4-amino-N-(4-pyridyl)thiochromane-7-carboxamide; (RS)-4-amino-N-(4-pyridyl)chromane-7-carboxamide; (RS)-5-amino-N-(4-pyridyl)-5,6,7,8-tetrahydronaphthalene-2-carboxamide; (RS)-5-amino-3-methyl-N-(4-pyridyl)-5,6,7,8-tetrahydronaphthalene-2-carboxamide; (RS)-3-amino-N-(4-pyridyl)-2,3-dihydrobenzo[b]thiophene-6-carboxamide 1,1-dioxide; (RS)-3-amino-5-methyl-N-(4-pyridyl)-2,3-dihydrobenzo[b]-thiophene-6-carboxamide 1,1-dioxide; (RS)-3-amino-N-(4-pyridyl)-2,3-dihydrobenzo[b]thiophene-6-carboxamide; (RS)-3-amino-5-methyl-N-(4-pyridyl)-2,3-dihydrobenzo[b]-thiophene-6-carboxamide; (RS)-3-amino-N-(4-pyridyl)-2,3-dihydrobenzo[b]furan-6-carboxamide; (RS)-1-amino-N-(4-pyridyl)indane-5-carboxamide; (RS)-5-amino-N-(4-pyridyl)-2,3,4,5-tetrahydro-1-benzothiepine-8-carboxamide 1,1-dioxide; (RS)-5-amino-N-(4-pyridyl)-2,3,4,5-tetrahydro-1-benzothiepine-8-carboxamide; (RS)-5-amino-N-(4-pyridyl)-2,3,4,5-tetrahydro-1-benzooxepine-8-carboxamide; (RS)-5-amino-N-(4-pyridyl)-6,7,8,9-tetrahydrobenzocycloheptene-2-carboxamide (RS)-4-amino-N-(1H-pyrrolo[2,3-b]pyridin-4-yl)thiochromane-7-carboxamide 1,1-dioxide; (RS)-4-amino-N-(1H-pyrrolo [2,3-b]pyridin-4-yl)thiochromane-7-carboxamide; (RS)-4-amino-N-(1H-pyrrolo[2,3-b]pyridin-4-yl)chromane-7-carboxamide; (RS)-5-amino-N-(1H-pyrrolo[2,3-b]pyridin-4-yl)-5,6,7,8-tetrahydronaphthalene-2-carboxamide; (RS)-5-amino-3-methyl-N-(1H-pyrrolo[2,3-b]pyridin-4-yl)-5,6,7,8-tetrahydronaphthalene-2-carboxamide; (RS)-3-amino-N-(1H-pyrrolo[2,3-b]pyridin-4-yl)-2,3-dihydrobenzo[b]thiophene-6-carboxamide 1,1-dioxide; (RS)-3-amino-5-methyl-N-(1H-pyrrolo[2,3-b]pyridin-4-yl)-2,3-dihydrobenzo[b]thiophene-6-carboxamide 1,1-dioxide; (RS)-3-amino-N-(1H-pyrrolo[2,3-b]pyridin-4-yl)-2,3-dihydrobenzo[b]thiophene-6-carboxamide; (RS)-3-amino-5-methyl-N-(1H-pyrrolo[2,3-b]pyridin-4-yl)-2,3-dihydrobenzo[b]thiophene-6-carboxamide; (RS)-3-amino-N-(1H-pyrrolo[2,3-b]pyridin-4-yl)-2,3-dihydrobenzo[b]furan-6-carboxamide; (RS)-1-amino-N-(1H-pyrrolo[2,3-b]pyridin-4-yl)indane-5-carboxamide; (RS)-5-amino-N-(1H-pyrrolo[2,3-b]pyridin-4-yl)-2,3,4,5-tetrahydro-1-benzothiepine-8-carboxamide 1,1-dioxide; (RS)-5-amino-N-(1H-pyrrolo[2,3-b]pyridin-4-yl)-2,3,4,5-tetrahydro-1-benzothiepine-8-carboxamide; (RS)-5-amino-N-(1H-pyrrolo[2,3-b]pyridin-4-yl)-2,3,4,5-tetrahydro-1-benzooxepine-8-carboxamide; (RS)-5-amino-N-(1H-pyrrolo[2,3-b]pyridin-4-yl)-6,7,8,9-tetrahydrobenzocyclo heptene-2-carboxamide; (RS)-4-amino-N-(1H-pyrazolo [3,4-b]pyridin-4-yl)thiochromane-7-carboxamide 1,1-dioxide; (RS)-4-amino-N-(1H-pyrazolo[3,4-b]pyridin-4-yl)thiochromane-7-carboxamide; (RS)-4-amino-N-(1H-pyrazolo[3,4-b]pyridin-4-yl)chromane-7-carboxamide; (RS)-5-amino-N-(1H-pyrazolo[3,4-b]pyridin-4-yl)-5,6,7,8-tetrahydronaphthalene-2-carboxamide; (RS)-5-amino-3-methyl-N-(1H-pyrazolo[3,4-b]pyridin-4-yl)-5,6,7,8-tetrahydronaphthalene-2-carboxamide; (RS)-3-amino-N-(1H-pyrazolo[3,4-b]pyridin-4-yl)-2,3-dihydrobenzo[b]thiophene-6-carboxamide 1,1-dioxide; (RS)-3-amino-5-methyl-N-(1H-pyrazolo[3,4-b]pyridin-4-yl)-2,3-dihydrobenzo[b]thiophene-6-carboxamide 1,1-dioxide; (RS)-3-amino-N-(1H-pyrazolo[3,4-b]pyridin-4-yl)-2,3-dihydrobenzo[b]thiophene-6-carboxamide; (RS)-3-amino-5-methyl-N-(1H-pyrazolo[3,4-b]pyridin-4-yl)-2,3-dihydrobenzo[b]thiophene-6-carboxamide; (RS)-3-amino-N-(1H-pyrazolo[3,4-b]pyridin-4-yl)-2,3-dihydrobenzo[b]furan-6-carboxamide; (RS)-1-amino-N-(1H-pyrazolo[3,4-b]pyridin-4-yl)indane-5-carboxamide; (RS)-5-amino-N-(1H-pyrazolo[3,4-b]pyridin-4-yl)-2,3,4,5-tetrahydro-1-benzothiepine-8-carboxamide 1,1-dioxide; (RS)-5-amino-N-(1H-pyrazolo[3,4-b]pyridin-4-yl)-2,3,4,5-tetrahydro-1-benzothiepine-8-carboxamide; (RS)-5-amino-N-(1H-pyrazolo[3,4-b]pyridin-4-yl)-2,3,4,5-tetrahydro-1-benzooxepine-8-carboxamide; (RS)-5-amino-N-(1H-pyrazolo[3,4-b]pyridin-4-yl)-6,7,8,9-tetrahydrobenzocycloheptene-2-carboxamide; (RS)-4-amino-8-methyl-N-(4-pyridyl)thiochromane-7-carboxamide; (RS)-4-amino-8-methyl-N-(4-pyridyl)thiochromane-7-carboxamide 1,1-dioxide; (RS)-4-amino-8-methyl-N-(1H-pyrazolo[3,4-b]pyridin-4-yl)thiochromane-7-carboxamide 1,1-dioxide; (RS)-4-amino-6-methyl-N-(4-pyridyl)thiochromane-7-carboxamide; (RS)-4-amino-6-methyl-N-(4-pyridyl)thiochromane-7-carboxamide 1,1-dioxide; (RS)-4-amino-6-methyl-N-(1H-pyrazolo[3,4-b]pyridin-4-yl)thiochromane-7-carboxamide; (RS)-4-amino-6-methyl-N-(1H-pyrazolo [3,4-b]pyridin-4-yl) thiochromane-7-carboxamide 1,1-dioxide; (RS)-4-amino-6-methyl-N-(1H-pyrrolo[2,3-b]pyridin-4-yl)thiochromane-7-carboxamide 1,1-dioxide; (RS)-4-amino-6-chloro-N-(4-pyridyl)thiochromane-7-carboxamide; (RS)-4-amino-6-chloro-N-(4-pyridyl)thiochromane-7-carboxamide 1,1-dioxide; (RS)-4-amino-6-chloro-N-(1H-pyrazolo[3,4-b]pyridin-4-yl)thiochromane-7-carboxamide; (RS)-4-amino-6-chloro-N-(1H-pyrazolo [3,4-b]pyridin-4-yl)thiochromane-7-carboxamide 1,1-dioxide; (RS)-4-amino-6-chloro-N-(1H-pyrrolo[2,3-b]pyridin-4-yl)thiochromane-7-carboxamide 1,1-dioxide; (RS)-4-amino-6-methoxy-N-(4-pyridyl)thiochromane-7-carboxamide; (RS)-4-amino-6-methoxy-N-(4-pyridyl)thiochromane-7-carboxamide 1,1-dioxide; (R)-4-amino-N-(4-pyridyl)thiochromane-7-carboxamide 1,1-dioxide; (R)-4-amino-N-(4-pyridyl)thiochromane-7-carboxamide; (R)-4-amino-N-(4-pyridyl)chromane-7-carboxamide; (R)-5-amino-N-(4-pyridyl)-5,6,7,8-tetrahydronaphthalene-2-carboxamide; (R)-5-amino-3-methyl-N-(4-pyridyl)-5,6,7,8-tetrahydronaphthalene-2-carboxamide; (R)-3-amino-N-(4-pyridyl)-2,3-dihydrobenzo[b]thiophene-6-carboxamide 1,1-dioxide; (R)-3-amino-5-methyl-N-(4-pyridyl)-2,3-dihydrobenzo[b]thiophene-6-carboxamide 1,1-dioxide; (R)-3-amino-N-(4-pyridyl)-2,3-dihydrobenzo[b]thiophene-6-carboxamide; (R)-3-amino-5-methyl-N-(4-pyridyl)-2,3-dihydrobenzo[b]-thiophene-6-carboxamide; (R)-3-amino-N-(4-pyridyl)-2,3-dihydrobenzo[b]furan-6-carboxamide; (R)-1-amino-N-(4-pyridyl)indane-5-carboxamide; (R)-5-amino-N-(4-pyridyl)-2,3,4,5-tetrahydro-1-benzothiepine-8-carboxamide 1,1-dioxide; (R)-5-amino-N-(4-pyridyl)-2,3,4,5-tetrahydro-1-benzothiepine-8-carboxamide; (R)-5-amino-N-(4-pyridyl)-2,3,4,5-tetrahydro-1-benzooxepine-8-carboxamide; (R)-5-amino-N-(4-pyridyl)-6,7,8,9-tetrahydrobenzocycloheptene-2-carboxamide; (R)-4-amino-N-(1H-pyrrolo[2,3-b]pyridin-4-yl)thiochromane-7-carboxamide 1,1-dioxide; (R)-4-amino-N-(1H-pyrrolo[2,3-b]pyridin-4-yl)thiochromane-7-carboxamide; (R)-4-amino-N-(1H-pyrrolo[2,3-b]pyridin-4-yl)chromane-7-carboxamide; (R)-5-amino-N-(1H-pyrrolo[2,3-b]pyridin-4-yl)-5,6,7,8-tetrahydronaphthalene -2-carboxamide; (R)-5-amino-3-methyl-N-(1H-pyrrolo[2,3-b]pyridin-4-yl)-5,6,7,8-tetrahydronaphthalene-2-carboxamide; (R)-3-amino-N-(1H-pyrrolo[2,3-b]pyridin-4-yl)-2,3-dihydrobenzo[b]thiophene-6-carboxamide 1,1-dioxide; (R)-3-amino-5-methyl-N-(1H-pyrrolo[2,3-b]pyridin-4-yl)-2,3-dihydrobenzo[b]thiophene-6-carboxamide 1,1-dioxide; (R)-3-amino-N-(1H-pyrrolo[2,3-b]pyridin-4-yl)-2,3-dihydrobenzo[b]thiophene-6-carboxamide; (R)-3-amino-5-methyl-N-(1H-pyrrolo[2,3-b]pyridin-4-yl)-2,3-dihydrobenzo[b]thiophene-6-carboxamide; (R)-3-amino-N-(1H-pyrrolo[2,3-b]pyridin-4-yl)-2,3-dihydrobenzo[b]furan-6-carboxamide; (R)-1-amino-N-(1H-pyrrolo[2,3-b]pyridin-4-yl)indane-5-carboxamide; (R)-5-amino-N-(1H-pyrrolo[2,3-b]pyridin-4-yl)-2,3,4,5-tetrahydro-1-benzothiepine-8-carboxamide 1,1-dioxide; (R)-5-amino-N-(1H-pyrrolo[2,3-b]pyridin-4-yl)-2,3,4,5-tetrahydro-1-benzothiepine-8-carboxamide; (R)-5-amino-N-(1H-pyrrolo[2,3-b]pyridin-4-yl)-2,3,4,5-tetrahydro-1-benzooxepine-8-carboxamide; (R)-5-amino-N-(1H-pyrrolo[2,3-b]pyridin-4-yl)-6,7,8,9-tetrahydrobenzocycloheptene-2-carboxamide; (R)-4-amino-N-(1H-pyrazolo[3,4-b]pyridin-4-yl)thiochromane-7-carboxamide 1,1-dioxide; (R)-4-amino-N-(1H-pyrazolo[3,4-b]pyridin-4-yl)thiochromane-7-carboxamide; (R)-4-amino-N-(1H-pyrazolo[3,4-b]pyridin-4-yl)chromane-7-carboxamide; (R)-5-amino-N-(1H-pyrazolo[3,4-b]pyridin-4-yl)-5,6,7,8-tetrahydronaphthalene-2-carboxamide; (R)-5-amino-3-methyl-N-(1H-pyrazolo[3,4-b]pyridin-4-yl)-5,6,7,8-tetrahydronaphthalene-2-carboxamide; (R)-3-amino-N-(1H-pyrazolo[3,4-b]pyridin-4-yl)-2,3-dihydrobenzo[b]thiophene-6-carboxamide 1,1-dioxide; (R)-3-amino-5-methyl-N-(1H-pyrazolo[3,4-b]pyridin-4-yl)-2,3-dihydrobenzo[b]thiophene-6-carboxamide 1,1-dioxide; (R)-3-amino-N-(1H-pyrazolo[3,4-b]pyridin-4-yl)-2,3-dihydrobenzo[b]thiophene-6-carboxamide; (R)-3-amino-5-methyl-N-(1H-pyrazolo[3,4-b]pyridin-4-yl)-2,3-dihydrobenzo[b]thiophene-6-carboxamide; (R)-3-amino-N-(1H-pyrazolo[3,4-b]pyridin-4-yl)-2,3-dihydrobenzo[b]furan-6-carboxamide; (R)-1-amino-N-(1H-pyrazolo [3,4-b]pyridin-4-yl)indane-5-carboxamide; (R)-5-amino-N-(1H-pyrazolo[3,4-b]pyridin-4-yl)-2,3,4,5-tetrahydro-1-benzothiepine-8-carboxamide 1,1-dioxide; (R)-5-amino-N-(1H-pyrazolo[3,4-b]pyridin-4-yl)-2,3,4,5-tetrahydro-1-benzothiepine-8-carboxamide; (R)-5-amino-N-(1H-pyrazolo[3,4-b]pyridin-4-yl)-2,3,4,5-tetrahydro-1-benzooxepine-8-carboxamide; (R)-5-amino-N-(1H-pyrazolo[3,4-b]pyridin-4-yl)-6,7,8,9-tetrahydrobenzocyclo heptene-2-carboxamide; (R)-4-amino-8-methyl-N-(4-pyridyl)thiochromane-7-carboxamide; (R)-4-amino-8-methyl-N-(4-pyridyl)thiochromane-7-carboxamide 1,1-dioxide; (R)-4-amino-8-methyl-N-(1H-pyrazolo[3,4-b]pyridin-4-yl)thiochromane-7-carboxamide 1,1-dioxide; (R)-4-amino-6-methyl-N-(4-pyridyl)thiochromane-7-carboxamide; (R)-4-amino-6-methyl-N-(4-pyridyl)thiochromane-7-carboxamide 1,1-dioxide; (R)-4-amino-6-methyl-N-(1H-pyrazolo[3,4-b]pyridin-4-yl)thiochromane-7-carboxamide; (R)-4-amino-6-methyl-N-(1H-pyrazolo[3,4-b]pyridin-4-yl)thiochromane-7-carboxamide 1,1-dioxide; (R)-4-amino-6-methyl-N-(1H-pyrrolo[2,3-b]pyridin-4-yl)thiochromane-7-carboxamide 1,1-dioxide; (R)-4-amino-6-chloro-N-(4-pyridyl)thiochromane-7-carboxamide; (R)-4-amino-6-chloro-N-(4-pyridyl)thiochromane-7-carboxamide 1,1-dioxide; (R)-4-amino-6-chloro-N-(1H-pyrazolo[3,4-b]pyridin-4-yl)thiochromane-7-carboxamide; (R)-4-amino-6-chloro-N-(1H-pyrazolo[3,4-b]pyridin-4-yl)thiochromane-7-carboxamide 1,1-dioxide; (R)-4-amino-6-chloro-N-(1H-pyrrolo [2,3-b]pyridin-4-yl)thiochromane-7-carboxamide 1,1-dioxide; (R)-4-amino-6-methoxy-N-(4-pyridyl)thiochromane-7-carboxamide; (R)-4-amino-6-methoxy-N-(4-pyridyl)thiochromane-7-carboxamide 1,1-dioxide; (S)-4-amino-N-(4-pyridyl)thiochromane-7-carboxamide 1,1-dioxide; (S)-4-amino-N-(4-pyridyl)thiochromane-7-carboxamide; (S)-4-amino-N-(4-pyridyl)chromane-7-carboxamide; (S)-5-amino-N-(4-pyridyl)-5,6,7,8-tetrahydronaphthalene-2-carboxamide; (S)-5-amino-3-methyl-N-(4-pyridyl)-5,6,7,8-tetrahydronaphthalene-2-carboxamide; (S)-3-amino-N-(4-pyridyl)-2,3-dihydrobenzo[b]thiophene-6-carboxamide 1,1-dioxide; (S)-3-amino-5-methyl-N-(4-pyridyl)-2,3-dihydrobenzo[b]-thiophene-6-carboxamide 1,1-dioxide; (S)-3-amino-N-(4-pyridyl)-2,3-dihydrobenzo[b]thiophene-6-carboxamide; (S)-3-amino-5-methyl-N-(4-pyridyl)-2,3-dihydrobenzo[b]-thiophene-6-carboxamide; (S)-3-amino-N-(4-pyridyl)-2,3-dihydrobenzo[b]furan-6-carboxamide; (S)-1-amino-N-(4-pyridyl)indane-5-carboxamide; (S)-5-amino-N-(4-pyridyl)-2,3,4,5-tetrahydro-1-benzothiepine-8-carboxamide 1,1-dioxide; (S)-5-amino-N-(4-pyridyl)-2,3,4,5-tetrahydro-1-benzothiepine-8-carboxamide; (S)-5-amino-N-(4-pyridyl)-2,3,4,5-tetrahydro-1-benzooxepine-8-carboxamide; (S)-5-amino-N-(4-pyridyl)-6,7,8,9-tetrahydrobenzocycloheptene-2-carboxamide; (S)-4-amino-N-(1H-pyrrolo[2,3-b]pyridin-4-yl)thiochromane-7-carboxamide 1,1-dioxide; (S)-4-amino-N-(1H-pyrrolo[2,3-b]pyridin-4-yl)thiochromane-7-carboxamide; (S)-4-amino-N-(1H-pyrrolo[2,3-b]pyridin-4-yl)chromane-7-carboxamide; (S)-5-amino-N-(1H-pyrrolo[2,3-b]pyridin-4-yl)-5,6,7,8-tetrahydronaphthalene -2-carboxamide; (S)-5-amino-3-methyl-N-(1H-pyrrolo[2,3-b]pyridin-4-yl)-5,6,7,8-tetrahydronaphthalene-2-carboxamide; (S)-3-amino-N-(1H-pyrrolo[2,3-b]pyridin-4-yl)-2,3-dihydrobenzo[b]thiophene-6-carboxamide 1,1-dioxide; (S)-3-amino-5-methyl-N-(1H-pyrrolo[2,3-b]pyridin-4-yl)-2,3-dihydrobenzo[b]thiophene-6-carboxamide 1,1-dioxide; (S)-3-amino-N-(1H-pyrrolo[2,3-b]pyridin-4-yl)-2,3-dihydrobenzo[b]thiophene-6-carboxamide; (S)-3-amino-5-methyl-N-(1H-pyrrolo[2,3-b]pyridin-4-yl)-2,3-dihydrobenzo[b]thiophene-6-carboxamide; (S)-3-amino-N-(1H-pyrrolo[2,3-b]pyridin-4-yl)-2,3-dihydrobenzo[b]furan-6-carboxamide;(S)-1-amino-N-(1H-pyrrolo[2,3-b]pyridin-4-yl)indane-5-carboxamide; (S)-5-amino-N-(1H-pyrrolo[2,3-b]pyridin-4-yl)-2,3,4,5-tetrahydro-1-benzothiepine-8-carboxamide 1,1-dioxide; (S)-5-amino-N-(1H-pyrrolo[2,3-b]pyridin-4-yl)-2,3 ,4,5-tetrahydro-1-benzothiepine-8-carboxamide; (S)-5-amino-N-(1H-pyrrolo[2,3-b]pyridin-4-yl)-2,3,4,5-tetrahydro-1-benzooxepine-8-carboxamide; (S)-5-amino-N-(1H-pyrrolo[2,3-b]pyridin-4-yl)-6,7,8,9-tetrahydrobenzocycloheptene-2-carboxamide; (S)-4-amino-N-(1H-pyrazolo[3,4-b]pyridin-4-yl)thiochromane-7-carboxamide 1,1-dioxide; (S)-4-amino-N-(1H-pyrazolo[3,4-b]pyridin-4-yl)thiochromane-7-carboxamide; (S)-4-amino-N-(1H-pyrazolo[3,4-b]pyridin-4-yl)chromane-7-carboxamide; (S)-5-amino-N-(1H-pyrazolo[3,4-b]pyridin-4-yl)-5,6,7,8-tetrahydronaphthalene-2-carboxamide; (S)-5-amino-3-methyl-N-(1H-pyrazolo[3,4-b]pyridin-4-yl)-5,6,7,8-tetrahydronaphthalene-2-carboxamide; (S)-3-amino-N-(1H-pyrazolo[3,4-b]pyridin-4-yl)-2,3-dihydrobenzo[b]thiophene-6-carboxamide 1,1-dioxide; (S)-3-amino-5-methyl-N-(1H-pyrazolo[3,4-b]pyridin-4-yl)-2,3-dihydrobenzo[b]thiophene-6-carboxamide 1,1-dioxide; (S)-3-amino-N-(1H-pyrazolo[3,4-b]pyridin-4-yl)-2,3-dihydrobenzo[b]thiophene-6-carboxamide; (S)-3-amino-5-methyl-N-(1H-pyrazolo[3,4-b]pyridin-4-yl)-2,3-dihydrobenzo[b]thiophene-6-carboxamide; (S)-3-amino-N-(1H-pyrazolo [3,4-b]pyridin-4-yl)-2,3-dihydrobenzo[b]furan-6-carboxamide; (S)-1-amino-N-(1H-pyrazolo[3,4-b]pyridin-4-yl)indane-5-carboxamide; (S)-5-amino-N-(1H-pyrazolo[3,4-b]pyridin-4-yl)-2,3,4,5-tetrahydro-1-benzothiepine-8-carboxamide 1,1-dioxide; (S)-5-amino-N-(1H-pyrazolo[3,4-b]pyridin-4-yl)-2,3,4,5-tetrahydro-1-benzothiepine-8-carboxamide; (S)-5-amino-N-(1H-pyrazolo[3,4-b]pyridin-4-yl)-2,3,4,5-tetrahydro-1-benzooxepine-8-carboxamide; (S)-5-amino-N-(1H-pyrazolo[3,4-b]pyridin-4-yl)-6,7,8,9-tetrahydrobenzocycloheptene-2-carboxamide; (S)-4-amino-8-methyl-N-(4-pyridyl)thiochromane-7-carboxamide; (S)-4-amino-8-methyl-N-(4-pyridyl)thiochromane-7-carboxamide 1,1-dioxide; (S)-4-amino-8-methyl-N-(1H-pyrazolo[3,4-b]pyridin-4-yl)thiochromane-7-carboxamide 1,1-dioxide; (S)-4-amino-6-methyl-N-(4-pyridyl)thiochromane-7-carboxamide; (S)-4-amino-6-methyl-N-(4-pyridyl)thiochromane-7-carboxamide 1,1-dioxide; (S)-4-amino-6-methyl-N-(1H-pyrazolo[3,4-b]pyridin-4-yl)thiochromane-7-carboxamide; (S)-4-amino-6-methyl-N-(1H-pyrazolo[3,4-b]pyridin-4-yl)thiochromane-7-carboxamide 1,1-dioxide; (S)-4-amino-6-methyl-N-(1H-pyrrolo[2,3-b]pyridin-4-yl)thiochromane-7-carboxamide 1,1-dioxide; (S)-4-amino-6-chloro-N-(4-pyridyl)thiochromane-7-carboxamide; (S)-4-amino-6-chloro-N-(4-pyridyl)thiochromane-7-carboxamide 1,1-dioxide; (S)-4-amino-6-chloro-N-(1H-pyrazolo[3,4-b]pyridin-4-yl)thiochromane-7-carboxamide; (S)-4-amino-6-chloro-N-(1H-pyrazolo[3,4-b]pyridin-4-yl)thiochromane-7-carboxamide 1,1-dioxide; (S)-4-amino-6-chloro-N-(1H-pyrrolo[2,3-b]pyridin-4-yl)thiochromane-7-carboxamide 1,1-dioxide; (S)-4-amino-6-methoxy-N-(4-pyridyl)thiochromane-7-carboxamide; (S)-4-amino-6-methoxy-N-(4-pyridyl) thiochromane-7-carboxamide 1,1-dioxide. Preferred are compounds having an (S)-configuration, more preferably the following compounds: (S)-4-amino-N-(4-pyridyl)thiochromane-7-carboxamide; (S)-4-amino-N-(4-pyridyl)thiochromane-7-carboxamide 1,1-dioxide; (S)-4-amino-6-methyl-N-(4-pyridyl)thiochromane-7-carboxamide; (S)-4-amino-6-methyl-N-(4-pyridyl)thiochromane-7-carboxamide 1,1-dioxide; (S)-4-amino-6-chloro-N-(4-pyridyl)thiochromane-7-carboxamide; (S)-4-amino-6-chloro-N-(4-pyridyl)thiochromane-7-carboxamide 1,1-dioxide; (S)-4-amino-8-methyl-N-(4-pyridyl)thiochromane-7-carboxamide; (S)-4-amino-8-methyl-N-(4-pyridyl)thiochromane-7-carboxamide 1,1-dioxide; (S)-4-amino-N-(1H-pyrazolo[3,4-b]pyridin-4-yl)thiochromane-7-carboxamide; (S)-4-amino-N-(1H-pyrazolo [3,4-b]pyridin-4-yl)thiochromane-7-carboxamide 1,1-dioxide; (S)-4-amino-N-(1H-pyrrolo[2,3-b]pyridin-4-yl)thiochromane-7-carboxamide; (S)-4-amino-N-(1H-pyrrolo[2,3-b]pyridin-4-yl)thiochromane-7-carboxamide 1,1-dioxide; (S)-4-amino-6-methyl-N-(1H-pyrrolo [2,3-b]pyridin-4-yl) thiochromane-7-carboxamide 1,1-dioxide; (S)-4-amino-6-chloro-N-(1H-pyrrolo[2,3-b]pyridin-4-yl)thiochromane-7-carboxamide 1,1-dioxide.

The compounds of the formula (III) and methods of their synthesis are disclosed in U.S. Pat. No. 6,933,305.

In some embodiments, the Rho kinase compound can comprise any of the compounds disclosed in columns 3-11 in U.S. Pat. No. 6,943,172 issued Sep. 13, 2005 to Nagarathnam et. al. incorporated herein by reference in its entirety. Methods of synthesis of such compounds are disclosed in the same patent.

In some embodiments, the Rho kinase compound can comprise any of the compounds disclosed on pages 4-14 of PCT publication No. 02/076976 published Oct. 3, 2002, which is incorporated herein by reference in its entirety. Methods of synthesizing these compounds are detailed in the same PCT publication. Particularly preferred compounds disclosed in PCT publication No. 02/076976 include 2-(2,4-dichlorophenyl)-N-(1H-indazol-5-yl)-4-quinazolinamine, 2-(4-chlorophenyl)-N-(1H-indazol-5-yl)-4-quinazolinamine, 1-{4-[4-(1H-indazol-5-ylamino)-2-quinazolinyl]phenyl}ethanone, N-(1H-indazol-5-yl)-2-[4-(trifluoromethyl)phenyl]-4-quinazolinamine, 2-(3-chloro-4-fluorophenyl)-N-(1H-indazol-5-yl)-4-quinazolinamine, 2-(1,3-benzodioxol-5-yl)-N-(1H-indazol-5-yl)-4-quinazolinamine, N-(1H-indazol-5-yl)-2-(4-methylphenyl)-4-quinazolinamine, 2-(3,4-dichlorophenyl)-N-(1H-indazol-5-yl)-4-quinazolinamine, N-(1H-indazol-5-yl)-2-(1-naphthyl)-4-quinazolinamine, N-(1H-indazol-5-yl)-2-(3,4,5-trimethoxyphenyl)-4-quinazolinamine, 2-(1-benzofuran-2-yl)-N-(1H-indazol-5-yl)-4-quinazolinamine, N-(1H-indazol-5-yl)-2-(2-thienyl)-4-quinazolinamine, N-(1H-indazol-5-yl)-2-(3-thienyl)-4-quinazolinamine, N-(1H-indazol-5-yl)-2-(3-methoxyphenyl)-4-quinazolinamine, N-(1H-indazol-5-yl)-2-(2-methoxyphenyl)-4-quinazolinamine, 2-(4-ethoxyphenyl)-N-(1H-indazol-5-yl)-4-quinazolinamine, 2-(3,5-dimethyl-4-isoxazolyl)-N-(1H-indazol-5-yl)-4-quinazolinamine, 2-(1,1′-biphenyl-4-yl)-N-(1H-indazol-5-yl)-4-quinazolinamine, 2-[4-(dimethylamino)phenyl]-N-(1H-indazol-5-yl)-4-quinazolinamine, 2-(1-benzothieN-2-yl)-N-(1H-indazol-5-yl)-4-quinazolinamine, N-(1H-indazol-5-yl)-2-(4-methoxyphenyl)-4-quinazolinamine, 4-[4-(1H-indazol-5-ylamino)-2-quinazolinyl]phenol, 2-dibenzo[b,d]furan-1-yl-N-(1H-indazol-5-yl)-4-quinazolinamine, 2-(2-fluoro-1,1′-biphenyl-4-yl)-N-(1H-indazol-5-yl)-4-quinazolinamine, 7-chloro-N-(1H-indazol-5-yl)-2-phenyl-4-quinazolinamine, N-(1H-indazol-5-yl)-6-nitro-2-phenyl-4-quinazolinamine, 2-(4-fluorophenyl)-N-(1H-indazol-5-yl)-6-nitro-4-quinazolinamine, 6-chloro-N-(1H-indazol-5-yl)-2-(4-methylphenyl)-4-quinazolinamine, 6-chloro-N-(1H-indazol-5-yl)-2-(4-methoxyphenyl)-4-quinazolinamine, 6-chloro-2-(4-fluorophenyl)-N-(1H-indazol-5-yl)-4-quinazolinamine, 6-chloro-N-(1H-indazol-5-yl)-2-(3-methoxyphenyl)-4-quinazolinamine, 2-(4-bromophenyl)-6-chloro-N-(1H-indazol-5-yl)-4-quinazolinamine, N-(1H-indazol-5-yl)-2-(2-quinoxalinyl)-4-quinazolinamine, 5-fluoro-N-(1H-indazol-5-yl)-2-(2-methylphenyl)-4-quinazolinamine, 5-fluoro-2-(4-fluorophenyl)-N-(1H-indazol-5-yl)-4-quinazolinamine, 2-(3-chlorophenyl)-5-fluoro-N-(1H-indazol-5-yl)-4-quinazolinamine, 2-(4-bromophenyl)-5-fluoro-N-(1H-indazol-5-yl)-4-quinazolinamine, 5-fluoro-N-(1H-indazol-5-yl)-2-(3-methylphenyl)-4-quinazolinamine hydrochloride, 2-(3-bromophenyl)-5-fluoro-N-(1H-indazol-5-yl)-4-quinazolinamine hydrochloride, 2-(2-chlorophenyl)-5-fluoro-N-(1H-indazol-5-yl)-4-quinazolinamine, 5-fluoro-N-(1H-indazol-5-yl)-2-(3-methoxyphenyl)-4-quinazolinamine bis(trifluoroacetate), 5-fluoro-N-(1H-indazol-5-yl)-2-(2-quinoxalinyl)-4-quinazolinamine tris(trifluoroacetate), 5-fluoro-N-(1H-indazol-5-yl)-2-(1-naphthyl)-4-quinazolinamine bis(trifluoroacetate), 5-fluoro-N-(1H-indazol-5-yl)-2-(2-naphthyl)-4-quinazolinamine bis(trifluoroacetate), 5-fluoro-N-(1H-indazol-5-yl)-2-(4-pyridinyl)-4-quinazolinamine tris(trifluoroacetate), N-(1H-indazol-5-yl)-7-methyl-2-(2-quinoxalinyl)-4-quinazolinamine, 2-(3-chlorophenyl)-N-(1H-indazol-5-yl)-7-methyl-4-quinazolinamine, 2-(4-fluorophenyl)-N-(1H-indazol-5-yl)-7-methyl-4-quinazolinamine, N-(1H-indazol-5-yl)-7-methyl-2-(4-methylphenyl)-4-quinazolinamine″, 2-(4-bromophenyl)-N-(1H-indazol-5-yl)-7-methyl-4-quinazolinamine, N-(1H-indazol-5-yl)-2-(4-methoxyphenyl)-7-methyl-4-quinazolinamine, N-(1H-indazol-5-yl)-7-methyl-2-(2-methylphenyl)-4-quinazolinamine bis(trifluoroacetate), N-(1H-indazol-5-yl)-7-methyl-2-(3-methylphenyl)-4-quinazolinamine bis(trifluoroacetate), N-[2-(3-fluorophenyl)-7-methyl-4-qu-inazolinyl]-N-(1H-indazol-5-yl)amine bis(trifluoroacetate), 2-(3-bromophenyl)-N-(1H-indazol-5-yl)-7-methyl-4-quinazolinamine bis(trifluoroacetate), N-[2-(2-chlorophenyl)-7-methyl-4-quinazolinyl]-N-(-1H-indazol-5-yl)amine bis(trifluoroacetate), N-(1H-indazol-5-yl)-2-(3-meth-oxyphenyl)-7-methyl-4-quinazolinamine bis(trifluoroacetate), 2-(3-furyl)-N-(1H-indazol-5-yl)-7-methyl-4-quinazolinamine bis(trifluoroacetate), N-(1H-indazol-5-yl)-7-methyl-2-(1-naphthyl)-4-quin-azolinamine bis(trifluoroacetate), N-(1H-indazol-5-yl)-7-methyl-2-(2-napht-hyl)-4-quinazolinamine bis(trifluoroacetate), N-(1H-indazol-5-yl)-7-methyl-2-(3-pyridinyl)-4-quinazolinamine tris(trifluoroacetate), N-(1H-indazol-5-yl)-7-methyl-2-(4-pyridinyl)-4-quinazolinamine tris(trifluoroacetate), 7-chloro-2-(3-chlorophenyl)-N-(1H-indazol-5-yl)-4-quinazolinamine, 7-chloro-N-(1H-indazol-5-yl)-2-(4-methylphenyl)-4-quinazolinamine, 2-(4-bromophenyl)-7-chloro-N-(1H-indazol-5-yl)-4-quinazolinamin-e, 7-chloro-N-(1H-indazol-5-yl)-2-(3-methylphenyl)-4-quinazolinamine hydrochloride, 7-chloro-2-(3-fluorophenyl)-N-(1H-indazol-5-yl)-4-quinazol-inamine bis(trifluoroacetate), 2-(3-bromophenyl)-7-chloro-N-(1H-indazol-5-yl)-4-quinazolinamine bis(trifluoroacetate), 7-chloro-N-(1H-indazol-5-yl)-2-(3-methoxyphenyl)-4-quinazolinamine bis(trifluoroacetate), N-[7-chloro-2-(2-furyl)-4-quinazolinyl]-N-(1H-indazol-5-yl)amine bis(trifluoroacetate), 7-chloro-N-(1H-indazol-5-yl)-2-(2-quinoxalinyl)-4-quinazolinamine tris(trifluoroacetate), 7-chloro-N-(1H-indazol-5-yl)-2-(1-naphthyl)-4-quinazolinamine bis(trifluoroacetate), 7-chloro-N-(1H-indazol-5-yl)-2-(2-naphthyl)-4-quinazolinamine bis(trifluoroacetate), 7-chloro-N-(1H-indazol-5-yl)-2-(3-pyridinyl)-4-quinazolinamine tris(trifluoroacetate), 2-(4-fluorophenyl)-N-(1H-indazol-5-yl)-6,7-dimeth-oxy-4-quinazolinamine, 2-(1,1′-biphenyl-4-yl)-N-(1H-indazol-5-yl)-6,7-dime-thoxy-4-quinazolinamine, N-(1H-indazol-5-yl)-6,7-dimethoxy-2-(3-methoxyphe-nyl)-4-quinazolinamine, N-(1H-indazol-5-yl)-6,7-dimethoxy-2-(4-vinylphenyl-)-4-quinazolinamine, 2-(4-ethoxyphenyl)-N-(1H-indazol-5-yl)-6,7-dimethoxy-4-quinazolinamine, N-cyclopentyl-4-(1H-indazol-5-ylamino)-2-quinazolinecar-boxamide, N-(3-fluorophenyl)-N-[4-(1H-indazol-5-ylamino)-6,7-dimethoxy-2-q-uinazolinyl]amine, N-(2,4-difluorobenzyl)-N-[4-(1H-indazol-5-ylamino)-6,7-dimethoxy-2-quinazolinyl]amine, N-(2-fluorobenzyl)-N-[4-(1H-indazol-5-ylam-ino)-6,7-dimethoxy-2-quinazolinyl]amine, N-(4-bromophenyl)-N-[4-(1H-indazo-1-5-ylamino)-6,7-dimethoxy-2-quinazolinyl]amine, N-(6,7-dimethoxy-2-{[4-(t-rifluoromethyl)phenyl]amino}-4-quinazolinyl)-N-(1H-indazol-5-yl)amine, N-(6,7-dimethoxy-2-{[4-(trifluoromethyl)benzyl]amino}-4-quinazolinyl)-N-(-1H-indazol-5-yl)amine, N-[3-fluoro-5-(trifluoromethyl)benzyl]-N-[4-(1H-ind-azol-5-ylamino)-6,7-dimethoxy-2-quinazolinyl]amine, N-(3-fluorobenzyl)-N-[4-(1H-indazol-5-ylamino)-6,7-dimethoxy-2-quinazolin-yl]amine, N-(2,4-difluorobenzyl)-N-[4-(1H-indazol-5-ylamino)-6,7-dimethoxy-2-quinazolinyl]amine, N-(4-fluorobenzyl)-N-[4-(1H-indazol-5-ylamino)-6,7-dimethoxy-2-quinazolinyl]amine, N-(2,6-difluorobenzyl)-N-[4-(1H-indazol-5-ylamino)-6,7-dimethoxy-2-quinazolinyl]amine, N-(3,5-difluorobenzyl)-N-[4-(-1H-indazol-5-ylamino)-6,7-dimethoxy-2-quinazolinyl]amine, N-(3-bromophenyl)-N-[4-(1H-indazol-5-ylamino)-6,7-dimethoxy-2-quinazoliny-1]amine, N-(2,6-difluorophenyl)-N-[4-(1H-indazol-5-ylamino)-6,7-dimethoxy-2-quinazolinyl]amine, N-(2,5-difluorophenyl)-N-[4-(1H-indazol-5-ylamino)-6- ,7-dimethoxy-2-quinazolinyl]amine, N-(2,4-difluorophenyl)-N-[4-(1H-indazol-5-ylamino)-6,7-dimethoxy-2-quinazolinyl]amine, N-(2,3-difluorophenyl)-N-[-4-(1H-indazol-5-ylamino)-6,7-dimethoxy-2-quinazolinyl]amine, N-(3,4-difluorophenyl)-N-[4-(1H-indazol-5-ylamino)-6,7-dimethoxy-2-quinaz-olinyl]amine, N-(3,5-difluorophenyl)-N-[4-(1H-indazol-5-ylamino)-6,7-dimet-hoxy-2-quinazolinyl]amine, N-{6,7-dimethoxy-2-[(2,3,4-trifluorophenyl)amin-o]-4-quinazolinyl}-N-(1H-indazol-5-yl)amine, N-{6,7-dimethoxy-2-[(2,4,5-tr-ifluorophenyl)amino]-4-quinazolinyl}-N-(1H-indazol-5-yl)amine, N-{6,7-dimethoxy-2-[(2,4,6-trifluorophenyl)amino]-4-quinazolinyl}-N-(1H-i-ndazol-5-yl)amine, N-{6,7-dimethoxy-2-[(2,3,6-trifluorophenyl)amino]-4-qui-nazolinyl}-N-(1H-indazol-5-yl)amine, N-(4-bromophenyl)-N-[4-(1H-indazol-5-ylamino)-6,7-dimethoxy-2-quinazolinyl]amine, 2-(3-aminophenyl)-N-(1H-indaz-ol-5-yl)-4-quinazolinamine, N-{3-[4-(1H-indazol-5-ylamino)-2-quinazolinyl]-phenyl}isonicotinamide, N-{3-[4-(1H-indazol-5-ylamino)-2-quinazolinyl]phen-yl}acetamide, N-(4-chlorophenyl)-N-[4-(1H-indazol-5-ylamino)-2-quinazoliny-1]amine, N-(3-bromophenyl)-N-[4-(1H-indazol-5-ylamino)-2-quinazolinyl]amin-e, N-(2-chlorophenyl)-N-[4-(1H-indazol-5-ylamino)-2-quinazolinyl]amine, N-(3-fluorophenyl)-N-[4-(1H-indazol-5-ylamino)-2-quinazolinyl]amine, N-(2-fluorophenyl)-N-[4-(1H-indazol-5-ylamino)-2-quinazolinyl]amine, N-(1H-indazol-5-yl)-N-{2-[(2-methoxyphenyl)amino]-4-quinazolinyl}amine, N-(1H-indazol-5-yl)-N-{2-[(3-methoxyphenyl)amino]-4-quinazolinyl}amine, N-(3-chlorophenyl)-N-[4-(1H-indazol-5-ylamino)-2-quinazolinyl]amine, N-(4-bromophenyl)-N-[4-(1H-indazol-5-ylamino)-2-quinazolinyl]amine, N-(1H-indazol-5-yl)-N-(2-{[3-(trifluoromethyl)phenyl]amino}-4-quinazoliny-l)amine, N-(1H-indazol-5-yl)-N-{2-[(4-phenoxyphenyl)amino]-4-quinazolinyl}-amine, N-(1H-indazol-5-yl)-N-(2-{[4-(trifluoromethoxy)phenyl]amino}-4-quin-azolinyl)amine, N-(1H-indazol-5-yl)-N-(2-{[3-(trifluoromethoxy)phenyl]amin-o}-4-quinazolinyl)amine, N-(4-fluorophenyl)-N-[4-(1H-indazol-5-ylamino)-2-quinazolinyl]amine, N-(2-anilino-4-quinazolinyl)-N-(1H-indazol-5-yl)amine, 2-[4-(2-chlorophenyl)-1-piperazinyl]-N-(1H-indazol-5-yl)-4-quinazolinamin-e, N-(1H-indazol-5-yl)-2-[4-(2-pyrimidinyl)-1-piperazinyl]-4-quinazolinami-ne, N-(1H-indazol-5-yl)-2-[4-(2-methoxyphenyl)-1-piperazinyl]-4-quinazolin-amine, 1-(4-{4-[4-(1H-indazol-5-ylamino)-2-quinazolinyl]-1-piperazinyl}phe-nyl)ethanone, 4-(1H-indazol-5-ylamino)-2-quinazolinecarboxamide″, 4-(1H-indazol-5-ylamino)-N-(4-pyridinyl)-2-quinazolinecarboxamide, 4-(1H-indazol-5-ylamino)-N-(4-methoxyphenyl)-2-quinazolinecarboxamide, N-cyclohexyl-4-(1H-indazol-5-ylamino)-2-quinazolinecarboxamide, N-cyclopentyl-4-(1H-indazol-5-ylamino)-2-quinazolinecarboxamide, 4-(1H-indazol-5-ylamino)-N-(2-pyridinyl)-2-quinazolinecarboxamide, 4-(1H-indazol-5-ylamino)-N-(3-quinolinyl)-2-quinazolinecarboxamide, 4-(1H-indazol-5-ylamino)-N-methyl-2-quinazolinecarboxamide, N-(1H-indazol-5-yl)-2-(4-morpholinylcarbonyl)-4-quinazolinamine, 2-(2,3-dihydro-1-benzofuran-5-yl)-N-(1H-indazol-5-yl)-4-quinazolinamine, 2-cyclopropyl-N-(1H-indazol-5-yl)-4-quinazolinamine, N-(1H-indazol-5-yl)-2-(trifluoromethyl)-4-quinazolinamine, N-(3-ethyl-1H-indazol-5-yl)-2-(4-methoxyphenyl)-4-quinazolinamine, 2-chloro-N-(3-ethyl-1H-indazol-5-yl)-4-quinazolinamine, 2-(2-fluoro-1,1′-biphenyl-4-yl)-N-(1H-indazol-5-yl)-4-quinazolinamine dihydrochloride, 2-(2-fluoro-1,1′-biphenyl-4-yl)-N-(1H-indazol-5-yl)-4-quinazolinamine dimethanesulfonate, 2-(2-fluoro-1,1′-biphenyl-4-yl)-N-(1H-indazol-5-yl)-4-quinazolinamine benzenesulfonate, 2-(2-fluoro-1,1′-biphenyl-4-yl)-N-(1H-indazol-5-yl)-4-q-uinazolinamine 4-methylbenzenesulfonate, and 2-dibenzo[b,d]furan-1-yl-N-(1-H-indazol-5-yl)-4-quinazolinamine trifluoroacetate, 2-chloro-N-(1H-indazol-5-yl)-4-quinazolinamine.

The compound to be used as the Rho kinase inhibitor may be a pharmaceutically acceptable acid addition salt. The acid is exemplified by inorganic acid such as hydrochloric acid, hydrobromic acid, sulfuric acid and the like and organic acid such as methanesulfonic acid, fumaric acid, maleic acid, mandelic acid, citric acid, tartaric acid, salicylic acid and the like. The compound having a carboxyl group can be converted to a salt with a metal such as sodium, potassium, calcium, magnesium, aluminum and the like or a salt with amino acid such as lysine and the like. In addition, their monohydrate, dihydrates, 1/2 hydrates, 1/3 hydrates, 1/4 hydrates, 2/3 hydrates, 3/2 hydrates and the like are also encompassed.

The Rho kinase inhibitor can be prepared as a general pharmaceutical agent. For example, the Rho kinase inhibitor can be mixed with a pharmaceutically acceptable carrier (e.g., excipient, binder, disintegrator, corrective, corrigent, emulsifier, diluent, solubilizer and the like) to give a pharmaceutical composition or a pharmaceutical preparation in the form of tablet, pill, powder, granule, capsule, troche, syrup, liquid, emulsion, suspension, injection (e.g., liquid, suspension and the like), suppository, inhalant, percutaneous absorber, eye drop, eye ointment and the like in the form suitable for oral or parenteral preparation.

When preparing a solid preparation, an additive such as sucrose, lactose, cellulose sugar, D-mannitol, maltitol, dextan, starches, agar, arginates, chitins, chitosans, pectines, tragacanth, gum arabic, gelatins, collagens, casein, albumin, calcium phosphate, sorbitol, glycine, carboxymethyl cellulose, polyvinylpyrrolidone, hydroxypropylcellulose, hydroxypropylmethylcellulose, glycerol, polyethyleneglycol, sodium hydrogencarbonate, magnesium stearate, talc and the like are used. Tablets can be applied with a typical coating, where necessary, to give sugar coated tablets, enteric tablets, film-coated tablets, two-layer tablets and multi-layer tablets.

When preparing a semi-solid preparation, animal and plant fats and oils (e.g., olive oil, corn oil, castor oil and the like), mineral fats and oils (e.g., petrolatum, white petrolatum, solid parain and the like), wax (e.g., jojoba oil, carnauba wax, bee wax and the like), partly or entirely synthesized glycerol fatty acid esters (e.g., lauric acid, myristic acid, palnitic acid and the like), and the like are used. Examples of commercially available products of these include Witepsol (manufactured by Dynamitnovel Ltd.), Farmazol (NOF Corporation) and the like.

When preparing a liquid preparation, an additive, such as sodium chloride, glucose, sorbitol, glycerol, olive oil, propylene glycol, ethyl alcohol and the like, is used. In particular, when preparing an injection, a sterile aqueous solution such as physiological saline, isotonizing liquid, oily liquid (e.g., sesame oil and soybean oil) and the like is used. Where necessary, a suitable suspending agent such as sodium carboxymethylcellulose, nonionic surfactant, solubilizer (e.g., benzyl benzoate and benzyl alcohol), and the like can be concurrently used. Moreover, when an eye drop is prepared, an aqueous liquid or solution is used, which is particularly a sterile injectable aqueous solution. The liquid for an eye drop can appropriately contain various additives such as buffer (preferred are borate buffer, acetate buffer, carbonate buffer and the like for less irritation), isotonizing agent, solubilizer, preservative, thickener, chelating agent, pH adjuster (preferably, pH is generally adjusted to about 6-8.5) and aromatic.

The content of the Rho kinase inhibitor in such preparations can be 0.1-100 wt %, suitably 1-50 wt %, of the preparation. While subject to variation depending on the condition, body weight, age and the like of patient, in general, about 1-500 mg of the active ingredient is orally administered daily for an adult in a single dose or several doses.

The Rho kinase inhibitor can be administered to a subject such as human by any appropriate method which includes oral administration, parenteral administration such as intravenous or subcutaneous administration and inhalation. The typical daily dose of the Rho-kinase inhibitor per se is disclosed, for example, in Abe K, Shimokawa H, Morikawa, et al. 2004. Circ Res. 94:385-93; Fukumoto Y, Matoba T, Ito A, et al. 2005. Heart. 91:391-92; Ishikura K, Yamada N, Ito M, et al. 2006. Circ J. 70:174-78; which are all incorporated herein by reference in their entirety. For example, total daily dose of Rho Kinase Inhibitor alone can range from 15 to 240 mg divided TID. When combined with Treprostinil, a lower dose of a Rho Kinase Inhibitor may be effective for treating pulmonary hypertension compared to administration the Rho Kinase Inhibitor alone.

In another aspect, the invention provides a kit comprising a first amount of treprostinil or its derivative or a pharmaceutically acceptable salt thereof, a second amount of a Rho kinase inhibitor or its derivative or a pharmaceutically acceptable salt thereof, and instructions for use in treating pulmonary hypertension, wherein the first amount and the second amount together form an amount effective for treatment pulmonary hypertension.

As used herein, the phrase “instructions for use” shall mean any FDA-mandated labeling, instructions, or package inserts that relate to the administration of Treprostinil or its derivatives, or a pharmaceutically acceptable salt thereof in combination with the administration of a Rho kinase inhibitor or its derivative or a pharmaceutically acceptable salt thereof, for treatment of pulmonary hypertension. For example, instructions for use may include, but are not limited to, indications for pulmonary hypertension, identification of specific symptoms associated with pulmonary hypertension, that can be ameliorated by a combination Treprostinil and Rho kinase inhibitor, and recommended dosage amounts for subjects suffering from pulmonary hypertension.

In some embodiments, the first amount of Treprostinil or its derivative or a pharmaceutically acceptable salt thereof can be contained in an inhalation device such as those described. In some embodiments, the first amount and the second amount can form a pharmaceutical composition for simultaneous administration to a subject in need thereof. In some embodiments, the first amount and the second amount of the kit are for separate or sequential administration to the subject.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A method of treating pulmonary hypertension comprising administering to a subject in need thereof
(A) a first amount of Treprostinil or its derivative, or a pharmaceutically acceptable salt thereof, and
(B) a second amount of a Rho Kinase inhibitor or its derivative or a pharmaceutically acceptable salt thereof,
wherein the first amount and the second amount together comprise an amount effective for treatment of pulmonary hypertension.

2. The method of claim 1, wherein said derivative of Treprostinil is an acid derivative of Treptostinil, a pro-drug of Treptostinil, a sustained release form of Treptostinil, an inhaled form of Treprostinil, an oral form of Treprostinil, a polymorph of Treprostinil or an isomer of Treprostinil.

3. The method of claim 1, wherein administering the first amount comprises administering a pharmaceutically acceptable salt of Treprostinil.

4. The method of claim 1, wherein administering the first amount comprises administering intravenously.

5. The method of claim 1, wherein administering the first amount comprises administering by inhalation.

6. The method of claim 5, wherein administering the first amount comprises administering using a nebulizer.

7. The method of claim 5, wherein administering the first amount comprises administering a metered dose inhaler.

8. The method of claim 7, wherein the metered dose inhaler is a soft mist inhaler.

9. The method of claim 1, wherein the Rho kinase inhibitor comprises a compound selected from the group consisting of 1-(5-isoquinolinesulfonyl)homopiperazide, 1-(1-hydroxy-5-isoquinolinesulfonyl)homopiperazine, (+)-trans-4-(1-aminoethyl)-1-(4-pyridylcarbamoyl)cyclohexane and pharmaceutically acceptable salts thereof.

10. The method of claim 9, wherein the Rho kinase inhibitor comprises a pharmaceutically acceptable salt of a compound selected from the group consisting of 1-(5-isoquinolinesulfonyl)homopiperazide, 1-(1-hydroxy-5-isoquinolinesulfonyl)homopiperazine, (+)-trans-4-(1-aminoethyl)-1-(4-pyridylcarbamoyl)cyclohexane.

11. The method of claim 1, wherein administering the second amount comprises administering the second amount orally.

12. The method of claim 1, wherein administering the second amount comprises administering the second amount parenterally.

13. The method of claim 10, wherein administering the second amount comprises administering the second amount intravenously.

14. The method of claim 10, wherein administering the second amount comprises administering the second amount subcutaneously.

15. The method of claim 1, wherein administering the second amount comprises administering the second amount by inhalation.

16. The method of claim 1, wherein administering the first amount and administering the second amount is performed separately, sequentially or simultaneously.

17. The method of claim 1, wherein the subject is a mammal.

18. The method of claim 17, wherein the subject is a human.

19. A kit for treating pulmonary hypertension comprising (i) a first amount of Treprostinil, or its derivative, or a pharmaceutically acceptable salt thereof, (ii) a second amount of a Rho kinase inhibitor or its derivative or a pharmaceutically acceptable salt thereof, and (iii) instructions for use in treating pulmonary hypertension, wherein the first amount and the second amount comprise together an amount effective for treatment of pulmonary hypertension.

20. The kit of claim 19, wherein said derivative is an acid derivative of Treptostinil, a pro-drug of Treptostinil, a sustained release form of Treptostinil, an inhaled form of Treprostinil, an oral form of Treprostinil, a polymorph of Treprostinil or an isomer of Treprostinil.

21. The kit of claim 19, the first amount comprises administering a pharmaceutically acceptable salt of Treprostinil.

22. The kit of claim 19, further comprising an inhalation device containing the first amount.

23. The kit of claim 22, wherein the inhalation device is a nebulizer.

24. The kit of claim 22, wherein the inhalation device is a metered dose inhaler.

25. The kit of claim 24, wherein the inhalation device is soft mist inhaler.

26. The kit of claim 19, wherein the Rho kinase inhibitor comprises a compound selected from the group consisting of 1-(5-isoquinolinesulfonyl)homopiperazide, 1-(1-hydroxy-5-isoquinolinesulfonyl)homopiperazine, (+)-trans-4-(1-aminoethyl)-1-(4-pyridylcarbamoyl)cyclohexane and pharmaceutically acceptable salts thereof.

27. The kit of claim 26, wherein the Rho kinase inhibitor comprises a pharmaceutically acceptable salt of a compound selected from the group consisting of 1-(5-isoquinolinesulfonyl)homopiperazide, 1-(1-hydroxy-5-isoquinolinesulfonyl)homopiperazine and (+)-trans-4-(1-aminoethyl)-1-(4-pyridylcarbamoyl)cyclohexane.

28. The kit of claim 19, wherein the second amount is in an oral formulation.

29. The kit of claim 19, wherein the second amount is in a parenteral formulation.

30. The kit of claim 29, wherein the second amount is in an intravenous formulation.

31. The kit of claim 29, wherein the second amount is in a subcutaneous formulation.

32. The kit of claim 19, wherein the second amount is in a formulation for inhalation.

33. The kit of claim 19, wherein the first amount and the second amount are for separate or sequential administration.
